(19) United States
(12) Patent Application Publication
(10) Pub. No.: US 2025/0002463 A1
(43) Pub. Date: Jan. 2, 2025

(54) NEW SALT FORMS OF AMILORIDE AND ITS DERIVATIVES FOR PHARMACEUTICAL USE

(71) Applicant: PSOMRI Holding AB, Nynäshamn (SE)

(72) Inventors: Giovanna Brancatelli, AB Oss (NL); Mårten Winge, Sunnyvale, CA (US); Rebecca Szafran, Lidingö (SE); Ilija Batljan, Nynäshamn (SE); Bruno Ekawa, AB Oss (NL)

(73) Assignee: PSOMRI Holding AB, Nynäshamn (SE)

(21) Appl. No.: 18/700,978

(22) PCT Filed: Sep. 28, 2022
(86) PCT No.: PCT/EP2022/077018

§ 371 Date: Apr. 12, 2024

(30) Foreign Application Priority Data
Sep. 28, 2021 (EP) 21199548.5

(51) Int. Cl. C07D 241/28; A61K 31/4965

(52) U.S. Cl. CPC C07D 241/28; A61K 31/4965

(57) ABSTRACT

The present invention relates to lactic, acetic, and phosphoric acid salts of amiloride and certain amiloride derivatives. The invention also relates to the use of such salts in medicine, pharmaceutical and cosmetic compositions comprising the novel salts, and to methods of treatment of psoriasis using the salts.

### FIELD OF THE INVENTION

The present invention relates to the field of pharmaceutical treatment, and in particular pharmaceutical treatment using new salts of certain amiloride derivatives.

### BACKGROUND

Amiloride (3,5-diamino-6-chloro-N-(diaminomethylidene)pyrazine-2-carboxamide, CAS registry number 2609-46-3) is commonly used, as the hydrochloride salt, as a potassium-sparing diuretic in the therapy of edema, often in combination with thiazide diuretics. Amiloride is known to interact with the epithelial sodium channel (ENaC) and acid-sensing ion channel proteins, as well as sodium/hydrogen antiporters (NHE) and sodium/calcium exchangers (NCX). Amiloride as the hydrochloride salt has been approved for marketing in Europe since at least the early 1970s.

One amiloride derivative is benzamil, or benzyl amiloride (3,5-diamino-N—(N′-benzylcarbamimidoyl)-6-chloropyrazine-2-carboxamide, CAS registry number 2898-76-2). WO2015/168574 suggests the use of epithelial ion channel blockers such as amiloride and its derivative benzamil in the treatment of psoriasis. WO2015/168574 discusses the use of pharmaceutically acceptable salts of amiloride and its derivatives, but does not mention specific salts or how to choose a specific salt. US 2013/109856 discloses a process of preparing the trifluoroacetic acid (TFA) salt of benzamil. The skilled person appreciates that TFA is not a suitable counterion for pharmaceutical purposes.

Psoriasis is an immune-mediated skin disease appearing in a chronic recurring manner. Prevalence estimates show that it affects 1-2% of the worldwide population with equal gender distribution. Psoriasis can emerge at any time of life and usually peaks between the ages of 30-39 and 60-69. Sufferers may experience itch, pain, and/or psoriasis-related nail disease and arthritis. Significant morbidity extends to the psychosocial impact on the individual. Psoriatic patients are often stigmatized by people staring at their disfigured skin; they may have low self-esteem and would face difficulties in relationships and employment.

Psoriasis has also been associated with an increased risk of cardiovascular diseases, stroke and cancer.

Histological assessment of psoriatic plaques demonstrates keratinocyte hyperproliferation with parakeratosis, epidermal elongation or rete ridges, increased angiogenesis, and dermal infiltration of inflammatory cells, including T cells, neutrophils, macrophages, and dendritic cells (DCs). Other histological features often observed in psoriatic skin include micropustules of Kogoj, microabscesses of Munro, thinned or absent granular layer, thinned suprapapillary plates, and the papillary dermis containing dilated superficial vessels.

The etiology of psoriasis is multifactorial. Environmental triggers, such as trauma, stress, infections and drugs, activate in predisposed individuals an exaggerated inflammatory response in the skin. Although psoriasis is a disease of dysfunctional proliferation and differentiation of the keratinocytes, there is significant T cell involvement through the release of inflammatory cytokines that promote further recruitment of immune cells, keratinocyte proliferation, and sustained chronic inflammation. These T-cells proliferate in the epidermis of psoriatic plaques.

The presence of innate immune cells and their products in psoriatic skin plaques indicates a role for innate immunity. Cells of the innate immune system include macrophages, NK and NKT cells, and DCs. There is an increased number of plasmacytoid and myeloid DCs in psoriatic skin compared with non-lesional skin. Other cellular elements of innate immunity are also involved in the development of psoriasis, including high numbers of macrophages which can secrete IL-6, IL-12, IL-23, and TNF. Keratinocytes are also capable resident antigen-presenting cells (APCs) in the skin. When stimulated they produce large amounts of cytokines (e.g., TNF, IL-6, and IL-18), chemotactic chemokines (e.g., IL-8 and CCL20), and antimicrobial peptides (e.g., β-defensin and LL37).

Genome wide scans have reported at least nine chromosomal loci linked to psoriasis, where the PSORS1 locus accounts for 35-50% of the heritability of the disease. PSORS1 is located on the major histological complex (MHC) region of chromosome 6 (6p21), and several genes contained within this region are thereby associated with psoriasis, namely, HLA-Cw6, CCHCR1 (coiled-coil o helical rod protein), and CDSN (corneodesmosin). Other susceptibility loci have been identified which include genes expressed in keratinocytes (LCE3B (late cornified envelope 3B) and LCE3C1 (late cornified envelope 3C1)) and immune cells (IL-12B, IL23R, and IL23A). This indicates that both the epidermal skin barrier and immune responses against pathogens are implicated in psoriasis pathogenesis.

Currently the first line of treatment for mild to moderate psoriasis is the use of topical agents. When topical therapy fails, escalated treatment often includes phototherapy, oral systemic agents, and/or injectable biological therapies. Corticosteroids, vitamin D analogues, and tazarotene all are used in the treatment of chronic plaque psoriasis. However, prolonged exposure to topical corticosteroids may lead to atrophy of the skin, permanent striae, and telangiectasia. Vitamin D analogues (e.g., calcitriol, calcipotriol, and tacalcitol) are effective antipsoriatic agents, but excessive use can lead to hypercalcemia. The probability of treatment success increases when combining vitamin D analogues with topical corticosteroids as compared with the vitamin D analogue monotherapy. As a result, an often recommended first-line induction treatment of plaque psoriasis is a combination of a vitamin D analogue and a topical steroid.

Other topical agents are commonly combined with topical corticosteroids and vitamin D analogues when treating psoriatic plaques. Salicylic acid is a topical keratolytic agent used adjunctly for removing scales, and it acts by reducing coherence between keratinocytes, increasing hydration, and softening of the stratum corneum by decreasing the skin pH. However, systemic salicylic acid toxicity can occur after long-term use over large skin areas. Retinoids, another popular treatment agent for psoriasis, act on skin by mediating or inducing cell differentiation and normalizing proliferation. Systemic retinoids, e.g. tazarotene, are associated with several adverse effects including teratogenicity, serum lipid elevations, mucocutaneous toxicity, skeletal changes, and hair loss.

Ultraviolet (UV) light therapy induces T-lymphocyte apoptosis in psoriatic lesions of the dermis and epidermis. Oral 8-methoxypsoralen-UV-A (PUVA) and narrowband UVB (NB-UVB) are well-established and effective treatments for chronic plaque psoriasis. PUVA has a response rate of approximately 80% compared with 70% for NB-UVB, however, NB-UVB is preferred because of higher convenience, except in case of very thick plaques. Systemic treatments are often used in combination with topical therapy and phototherapy for patients with severe psoriasis. Oral systemic agents for the treatment of psoriasis include methotrexate, cyclosporine, and acitretin. Injectable biological therapies are emerging approaches for the treatment of psoriasis by targeting molecules in the inflammatory pathways. They are considered for patients with severe psoriasis that are resistant to oral immunosuppressants and phototherapy. The two major therapeutic classes of injectable biological therapies include anti-cytokine therapies and T-cell-targeted therapies. The first class consists of injectable immunoglobulins (Ig), infliximab, and adalimumab, target soluble and membrane-bound TNF. Other anti-cytokine therapies include etanercept and ustekinumab. A second therapeutic class of injectable therapies include agents that bind to T cells and prevent T-cell activation, including alefacept and efalizumab.

Dermatologists and patients would benefit from new therapies for psoriasis, both those that can be delivered topically as well as those that can be delivered systemically.

### SUMMARY

The present inventors have found that certain salts of the amiloride derivative benzamil have improved properties at least in terms of improved stability, increased water solubility, and/or reduced polymorphism as compared to the free base compound and known salts of benzamil.

The present invention thus provides novel salt forms of benzamil, amiloride and certain amiloride derivatives.

Free base compounds useful in the present invention are those of formula (I)

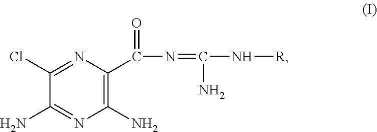

wherein R is selected from

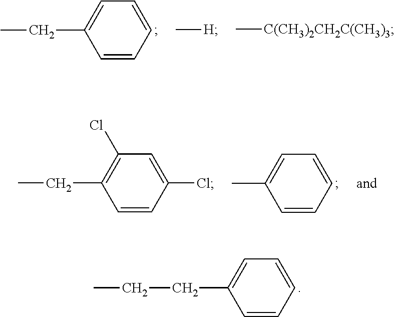

According to a first aspect there is provided a salt of the free base above, which is a lactic acid salt, an acetic acid salt, or a phosphoric acid salt.

In one embodiment, the salt is the lactic acid salt.

In one embodiment, the free base compound is benzamil.

In one embodiment, the salts are in only one crystal form.

In a second aspect, the present invention relates to a pharmaceutical or cosmetic composition comprising the salt according to the invention, and optionally pharmaceutically and/or cosmetically acceptable excipients.

In one embodiment, the pharmaceutical composition is adapted for topical administration.

In a third aspect, the present invention relates to the salt according to the first aspect, or the pharmaceutical composition according to the second aspect, for use in medicine.

In one embodiment, the present invention relates to the salt or pharmaceutical composition for use in a method of treatment of psoriasis.

In one embodiment, the psoriasis is chronic psoriasis or plaque psoriasis.

In one embodiment, the salt or composition is administered topically or systemically.

In a fourth aspect, the present invention relates to the use of the salt according to the first aspect in the manufacture of a pharmaceutical composition for use in in a method of treatment of psoriasis.

In one embodiment, the psoriasis is chronic psoriasis or plaque psoriasis.

In one embodiment, the salt is administered topically or systemically.

In a fifth aspect, the present invention relates to a method for treatment of psoriasis comprising administering to an individual in need thereof an effective amount of a salt according to the first aspect, or of a pharmaceutical composition according to the second aspect.

In one embodiment, the psoriasis is chronic psoriasis or plaque psoriasis.

In one embodiment, the salt is administered topically or systemically.

### BRIEF DESCRIPTION OF THE FIGURES

FIG. 1 shows a High Throughput XRPD diffractogram for the form LAC1 of the lactic acid salt of benzamil.

FIG. 2 shows a High Throughput XRPD diffractogram for the form LAC1 of the lactic acid salt of benzamil as prepared and after submission to accelerated Aging Conditions (AAC).

FIG. 3 shows a High Throughput XRPD diffractogram for the form ACA1 of the acetic acid salt of benzamil.

FIG. 4 shows a High Throughput XRPD diffractogram for the form ACA2 of the acetic acid salt of benzamil.

FIG. 5. shows a High Throughput XRPD diffractogram for the forms ACA1 and ACA2 of the acetic acid salt of benzamil as prepared and after submission to accelerated Aging Conditions (AAC).

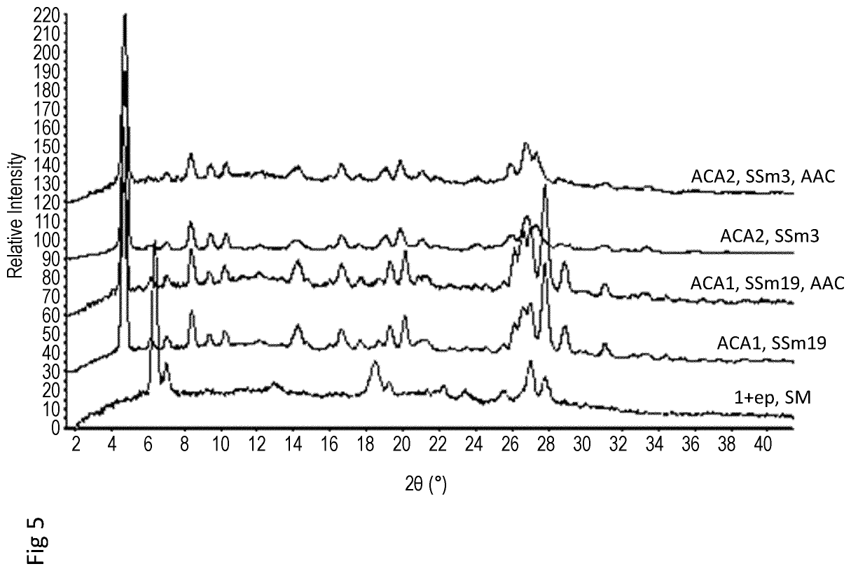

FIG. 6 shows a High Throughput XRPD diffractogram for the form PHO2 of the phosphoric acid salt of benzamil.

FIG. 7 shows a High Throughput XRPD diffractogram for the form PHO2 of the phosphoric acid salt of benzamil as prepared and after submission to accelerated Aging Conditions (AAC).

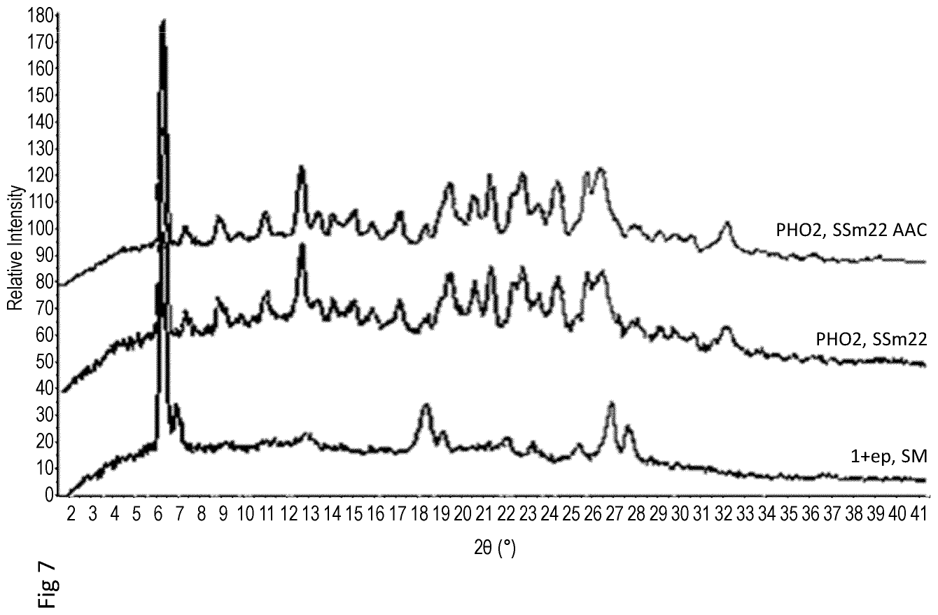

FIG. 8 shows intrinsic dissolution rate profiles in water of A) benzamil lactate (LAC1) and B) benzamil TFA (TFA2).

### DEFINITIONS

Unless defined otherwise, all technical and scientific terms used herein have the same meaning as commonly understood to one of ordinary skill in the art to which this invention belongs.

The term “sensitivity” and “sensitive” when made in reference to treatment is a relative term which refers to the degree of effectiveness of a treatment compound in lessening or decreasing the symptoms of the disease being treated. For example, the term “increased sensitivity” when used in reference to treatment of a cell or patient refers to an increase of at least 5% or more, in the effectiveness in lessening or decreasing the symptoms of psoriasis when measured using any methods well-accepted in the art.

As used herein, and unless otherwise specified, the term “therapeutically effective amount” of a compound is an amount sufficient to provide a therapeutic benefit in the treatment or management of psoriasis, or to delay or minimize one or more symptoms associated with psoriasis. A therapeutically effective amount of a compound means an amount of therapeutic agent, alone or in combination with other therapies, which provides a therapeutic benefit in the treatment or management of psoriasis. The term “therapeutically effective amount” can encompass an amount that improves overall therapy, reduces or avoids symptoms or causes of psoriasis, or enhances the therapeutic efficacy of another therapeutic agent.

The term “likelihood” generally refers to an increase in the probability of an event. The term “likelihood” when used in reference to the effectiveness of a patient response generally contemplates an increased probability that the symptoms of psoriasis will be lessened or decreased.

The terms “determining”, “measuring”, “evaluating”, “assessing” and “assaying” as used herein generally refer to any form of measurement, and include determining if an element is present or not. These terms include both quantitative and/or qualitative determinations. Assessing may be relative or absolute. “Assessing the presence of” can include determining the amount of something present, as well as determining whether it is present or absent.

The term “sample” as used herein relates to a material or mixture of materials, typically, although not necessarily, in fluid form, containing one or more components of interest.

“Biological sample” as used herein refers to a sample obtained from a biological subject, including sample of biological tissue or fluid origin, obtained, reached, or collected in vivo or in situ. A biological sample also includes samples from a region of a biological subject containing precancerous or cancer cells or tissues. Such samples can be, but are not limited to, organs, tissues, fractions and cells isolated from a mammal. Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like. Preferred biological samples include but are not limited to whole blood, partially purified blood. PBMCs, tissue biopsies, and the like.

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, includes methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at separate times, so long as the first agent (and additional agents) are after administration in the presence of the second agent (and additional agents). The actor and the subject may be the same entity (e.g., human).

As used herein, the term “dose amount” refers to the quantity, e.g., milligrams (mg), of the substance which is administered to the subject. In one embodiment, the dose amount is a fixed dose, e.g., is not dependent on the weight of the subject to which the substance is administered. In another embodiment, the dose amount is a relative and not fixed dose, e.g., is dependent on the weight of the subject to which the substance is administered, or for a topical therapy a dose may be related to the surface area that is treated, e.g. dose/m² of skin.

As used herein, the term “periodicity” as it relates to the administration of a substance refers to a (regular) recurring cycle of administering the substance to a subject.

The “duration of a periodicity” refers to a time over which the recurring cycle of administration occurs.

As used herein, and unless otherwise specified, the terms “treat,” “treating” and “treatment” refer to an action that occurs while a patient is suffering from psoriasis, which reduces the severity of psoriasis, or retards or slows the progression of the psoriasis, or achieves or maintains a therapeutic objective. An “effective patient response” refers to any increase in the therapeutic benefit to the patient. An “effective patient psoriasis response” can be, for example, a 5%, 10%, 25%, 50%, or 100% decrease in the physical symptoms of psoriasis.

The term “kit” as used herein refers to a packaged product comprising components with which to administer the novel salt form of the invention for treatment of psoriasis. The kit preferably comprises a box or container that holds the components of the kit. The box or container may be affixed with a label or a Food and Drug Administration approved protocol. The box or container holds components of the invention which are preferably contained within plastic, polyethylene, polypropylene, ethylene, or propylene vessels. The vessels can be capped-tubes or bottles. The kit can also include instructions for use.

The phrase “pharmaceutically acceptable” is employed herein to refer to those compounds, materials, compositions, and/or dosage forms which are, within the scope of sound medical judgment, suitable for use in contact with the tissues of the subject with toxicity, irritation, allergic response, or other problems or complications, commensurate with a reasonable benefit/risk ratio. Compounds, materials, compositions, and/or dosage forms that are pharmaceutically acceptable are also considered cosmetically acceptable.

The phrase “pharmaceutically acceptable excipient” as used herein refers to an acceptable material, composition or vehicle, such as a liquid or solid filler, diluent, carrier, manufacturing aid (e.g., lubricant, talc, magnesium, calcium or zinc stearate, or steric acid), solvent or encapsulating material, involved in carrying or transporting the, optionally therapeutic, compound for administration to the subject. Each excipient should be “acceptable” in the sense of being compatible with the other ingredients of the formulation and not injurious to the subject. Some examples of materials which can serve as pharmaceutically excipients include: ethanol, sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations. If desired, certain sweetening and/or flavoring and/or coloring agents may be added. Pharmaceutically acceptable excipients are also considered cosmetically acceptable excipients.

The term “compound” as used herein includes compounds in free base from as well as in salt forms.

PSM001, benzyl amiloride and benzamil are used interchangeably and are all intended to refer to 3,5-diamino-N—(N′-benzylcarbamimidoyl)-6-chloropyrazine-2-carboxamide (CAS registry number 2898-76-2).

### DETAILED DESCRIPTION

The present invention provides novel salts of amiloride, and of amiloride derivatives, that have improved properties at least in terms of improved stability, increased water solubility, and/or reduced polymorphism.

According to a first aspect, the invention relates to a salt of a free base compound represented by formula (I)

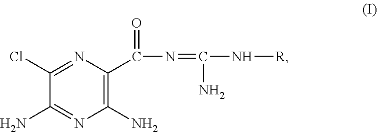

wherein the salt is selected from the lactic acid salt, acetic acid salt, and phosphoric acid salt.

A salt according to the present invention may have an increased water solubility, compared to salts of the prior art. The skilled formulator appreciates that an increased water solubility is advantageous when preparing an active pharmaceutical ingredient (API) formulation. For example, a higher concentration of the API may be used which may provide a higher dose in a formulation. Such formulation may be a topical formulation or a formulation intended for systemic delivery.

In one embodiment, R is benzyl, i.e. the free base compound is benzamil.

In one embodiment the salt is the lactic acid salt. An advantage with lactate as a counterion in a pharmaceutical salt is that it is endogenous, and is present for example in the skin of a human. This is particularly advantageous in topical formulations. Another advantage with lactic acid is that it is a keratolytic agent. Thus, in embodiments wherein the salt is a lactic acid salt, both the free base and the counterion may have a therapeutic effect. Thus, a formulation comprising a free base compound of formula (I) in combination with a lactate salt may provide for a formulation with a combination effect, suitable for a combination treatment.

In one embodiment the salt is the acetic acid salt.

In one embodiment the salt is the phosphoric acid salt.

In one embodiment, the salt is in only one crystal form. The skilled formulator appreciates that the presence of a single polymorph may be advantageous for formulations of an API. For example, API's in different polymorphs may have different release rates. Moreover, a ratio of the different polymorphs in a formulation may change over time. This may lead to an undesired change in release rate over time in a formulation of a salt having more than one polymorph.

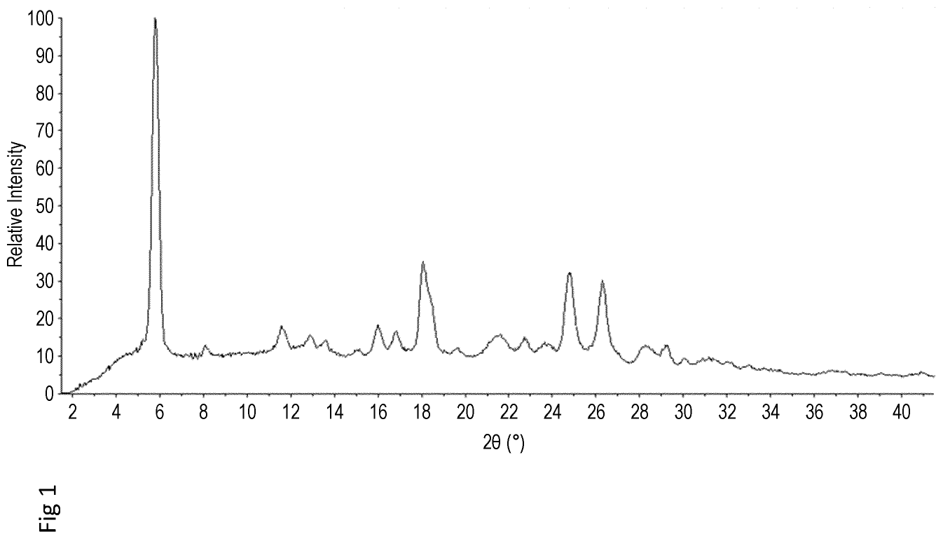

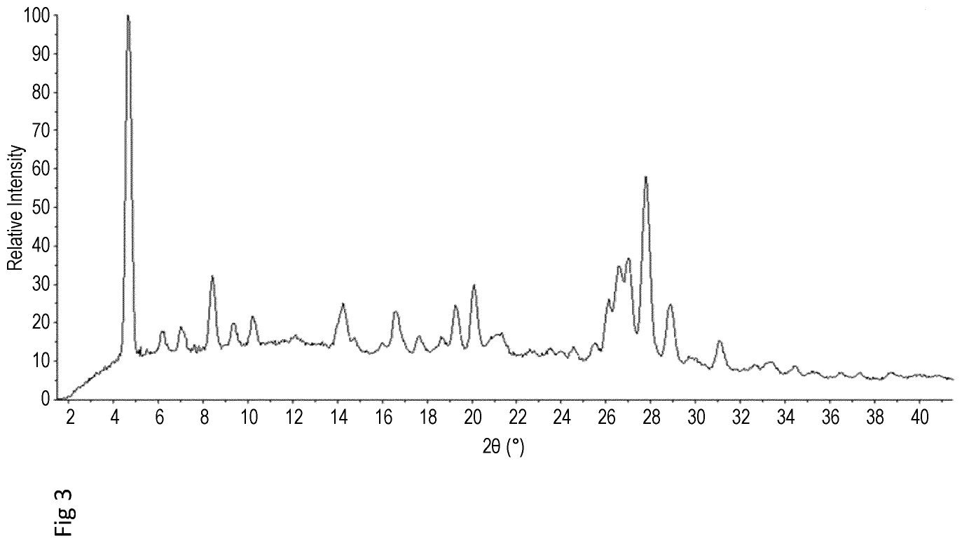

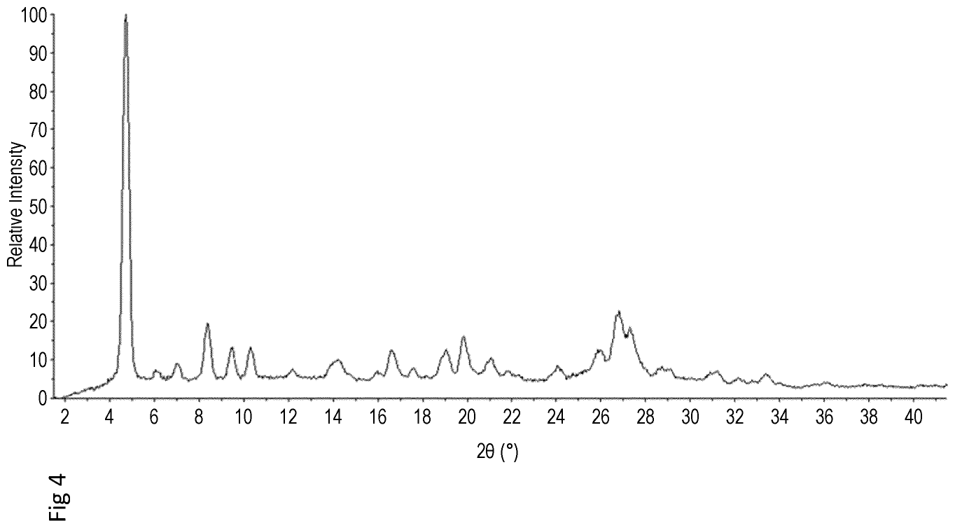

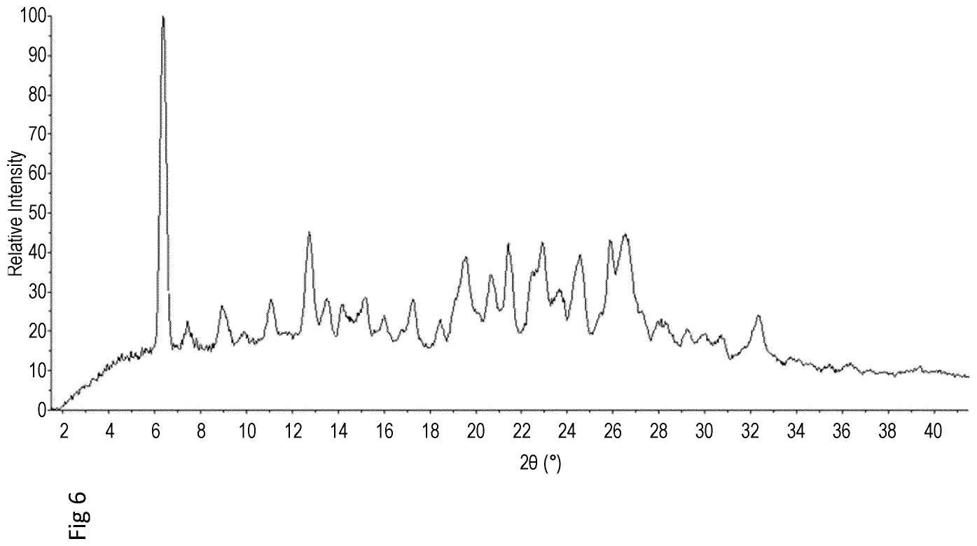

In one embodiment, the free base compound is benzamil, the salt is the lactic acid salt, and the X-ray powder diffractogram of the salt comprises characteristic peaks as shown in FIG. 1. In one embodiment, the free base compound is benzamil, the salt is the acetic acid salt, and the X-ray powder diffractogram of the salt comprises characteristic peaks as shown in FIG. 3. In one embodiment, the free base compound is benzamil, the salt is the acetic acid salt, and the X-ray powder diffractogram of the salt comprises characteristic peaks as shown in FIG. 4. In one embodiment, the free base compound is benzamil, the salt is the phosphoric acid salt, and the X-ray powder diffractogram of the salt comprises characteristic peaks as shown in FIG. 6.

In one embodiment, R is hydrogen, i.e. the free base compound is amiloride. In one embodiment the salt is the lactic acid salt. In one embodiment the salt is the acetic acid salt.

In one embodiment the salt is the phosphoric acid salt. In one embodiment, the salt is in only one crystal form.

In one embodiment, R is —C(CH₃)₂CH₂C(CH₃)₃. In one embodiment the salt is the lactic acid salt. In one embodiment the salt is the acetic acid salt. In one embodiment the salt is the phosphoric acid salt. In one embodiment, the salt is in only one crystal form.

In one embodiment, R is

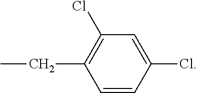

In one embodiment the salt is the lactic acid salt. In one embodiment the salt is the acetic acid salt. In one embodiment the salt is the phosphoric acid salt. In one embodiment, the salt is in only one crystal form.

In one embodiment, R is phenyl, i.e. the free base compound is phenamil. In one embodiment the salt is the lactic acid salt. In one embodiment the salt is the acetic acid salt. In one embodiment the salt is the phosphoric acid salt. In one embodiment, the salt is in only one crystal form.

In one embodiment, R is

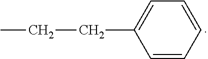

In one embodiment the salt is the lactic acid salt. In one embodiment the salt is the acetic acid salt. In one embodiment the salt is the phosphoric acid salt. In one embodiment, the salt is in only one crystal form.

### Pharmaceutical Use of the Novel Salts

The therapeutic effect of amiloride as a potassium-sparing diuretic is well established. The therapeutic effect of benzamil on psoriasis has been plausibly shown in the examples of published PCT application WO2015168574, incorporated herein by reference.

WO2015168574 furthermore discloses that benzamil targets the epithelial sodium channel (ENaC) and sodium/calcium exchangers (NCX1) in human psoriatic keratinocytes. The present inventors have realized that related compounds with potency similar to benzamil on ENaC, NCX1, and also the Na⁺/H⁺ exchanger NHE, are likely to provide similar advantageous effects as benzamil. Such compounds are disclosed in the prior art (Kleyman et al (1988), J Membrane Biol, 105:1-21), and include the free base compounds used in the present invention. Therapeutic efficacy can be plausibly established using the experimental protocol provided in WO2015168574.

The present invention thus in certain aspects relates to novel salts of benzamil, amiloride, and certain other amiloride derivatives for use in medicine.

In certain aspects, the present invention relates to novel salts of benzamil, amiloride, and certain other amiloride derivatives for use in the treatment of psoriasis. In embodiments, the form of psoriasis to be treated is any of the forms further discussed below.

Chronic plaque psoriasis (also referred to as psoriasis vulgaris) is the most common form of psoriasis. Chronic plaque psoriasis is characterized by raised reddened patches of skin, ranging from coin-sized to much larger. In chronic plaque psoriasis, the plaques may be single or multiple, they may vary in size from a few millimeters to several centimeters. The plaques are usually red with a scaly surface, and reflect light when gently scratched, creating a “silvery” effect. Lesions (which are often symmetrical) from chronic plaque psoriasis occur all over body, but with predilection for extensor surfaces, including the knees, elbows, lumbosacral regions, scalp, and nails. Occasionally chronic plaque psoriasis can occur on the penis, vulva and flexures, but scaling is usually absent. Diagnosis of patients with chronic plaque psoriasis is usually based on the clinical features described above. In particular, the distribution, color and typical silvery scaling of the lesion in chronic plaque psoriasis are characteristic of chronic plaque psoriasis.

Guttate psoriasis refers to a form of psoriasis with characteristic water drop shaped scaly plaques. Flares of guttate psoriasis generally follow an infection, most notably a streptococcal throat infection. Diagnosis of guttate psoriasis is usually based on the appearance of the skin, and the fact that there is often a history of recent sore throat.

Inverse psoriasis is a form of psoriasis in which the patient has smooth, usually moist areas of skin that are red and inflamed, which is unlike the scaling associated with plaque psoriasis. Inverse psoriasis is also referred to as intertiginous psoriasis or flexural psoriasis. Inverse psoriasis occurs mostly in the armpits, groin, under the breasts and in other skin folds around the genitals and buttocks, and, as a result of the locations of presentation, rubbing and sweating can irritate the affected areas.

Pustular psoriasis, also referred to as palmar plantar psoriasis, is a form of psoriasis that causes pus-filled blisters that vary in size and location, but often occur on the hands and feet. The blisters may be localized, or spread over large areas of the body. Pustular psoriasis can be both tender and painful, can cause fevers.

Erythroderma psoriasis is a particularly inflammatory form of psoriasis that often affects most of the body surface. It may occur in association with von Zumbusch pustular psoriasis. It is a rare type of psoriasis, occurring once or more during the lifetime of 3 percent of people who have psoriasis. It generally appears on people who have unstable plaque psoriasis. Widespread, fiery redness and exfoliation of the skin characterize this form. Severe itching and pain often accompany this manifestation. Erythrodermic psoriasis causes protein and fluid loss that can lead to severe illness. Edema (swelling from fluid retention), especially around the ankles, may develop, along with infection. Erythrodermic psoriasis also can bring on pneumonia and congestive heart failure. People with severe cases often require hospitalization. Erythrodermic psoriasis can occur abruptly at the first signs of psoriasis or it can come on gradually in people with plaque psoriasis. Combination treatments are frequently required, for example topical products and one or two systemic medications.

The present invention thus also relates to and makes use of pharmaceutically acceptable compositions which comprise a therapeutically-effective amount of at least one salt according to the invention, optionally combined with one or more additional agents for treatment of psoriasis, formulated together with one or more pharmaceutically acceptable excipients. The active ingredients and excipient(s) may be formulated into compositions and dosage forms according to methods known in the art. The pharmaceutical compositions of the present invention may be formulated for administration via topical application, for example, as a lotion, cream, ointment, spray, patch, microneedle array, etc. applied to the skin; in solid, liquid or semi-liquid form, including those adapted for the following: oral administration, for example, tablets, capsules, powders, granules, pastes for application to the tongue, aqueous or non-aqueous solutions or suspensions, drenches, or syrups.

The present invention thus also relates to and makes use of cosmetically acceptable compositions which comprise an amount of at least one salt according to the invention, optionally combined with one or more additional agents, formulated together with one or more acceptable excipients. Cosmetic compositions may be useful in improving appearance or feel of the skin of a subject, while not having an effect on the skin that is considered therapeutic. The salt and excipient(s) may be formulated into compositions and dosage forms according to methods known in the art. The cosmetic compositions of the present invention may be formulated for administration via topical application, for example, as a lotion, cream, ointment, spray, patch, microneedle array, etc. applied to the skin; in solid, liquid or semi-liquid form, including those adapted for the following: oral administration, for example, tablets, capsules, powders, granules, pastes for application to the tongue, aqueous or non-aqueous solutions or suspensions, drenches, or syrups.

The medicaments, pharmaceutical compositions, or therapeutic combinations according to the present invention may be in any form suitable for the application to humans and/or animals, preferably humans including infants, children and adults, and can be produced by standard procedures known to those skilled in the art. The medicament, (pharmaceutical) composition or therapeutic combination can be produced by standard procedures known to those skilled in the art, e.g. from the table of contents of “Pharmaceutics: The Science of Dosage Forms”, Second Edition, Aulton, M. E. (ED. Churchill Livingstone, Edinburgh (2002); “Encyclopedia of Pharmaceutical Technology”, Second Edition, Swarbrick, J. and Boylan J. C. (Eds.), Marcel Dekker, Inc. New York (2002); “Modern Pharmaceutics”, Fourth Edition, Banker G. S. and Rhodes C. T. (Eds.) Marcel Dekker, Inc. New York 2002 y “The Theory and Practice of Industrial Pharmacy”, Lachman L., Lieberman H. And Kanig J. (Eds.), Lea & Febiger, Philadelphia (1986). The respective descriptions are hereby incorporated by reference and form part of the disclosure.

An effective dose of the salt according to the invention may include a “therapeutically effective dose or amount” or a “prophylactically effective dose or amount” as defined above. A therapeutically effective amount may vary according to factors such as the disease state, age, sex, and weight of the individual, and the ability to elicit a desired response in the individual. A therapeutically effective dose/amount is also one in which any toxic or detrimental effects are outweighed by the therapeutically beneficial effects. A “prophylactically effective dose/amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Treatment of psoriasis may entail achieving or maintaining a PGA score of 0/1 or a PASI 50, PASI 75, PASI 90, or PASI 100 response score for a period of time during or following treatment (e.g., for at least 2, 4, 6, 8, 10, 12, 14, 16, 18, 20, 22, 24, 26, 28, 30, 32, 34, 36, 38, 40, 42, 46, 48, 50, 52, 54, 56, 58 or 60 weeks or longer). Treatment of psoriasis may also entail achieving or maintaining a health-related quality of life (HRQOL) outcome. HRQOL outcomes include Dermatology Life Quality Index (DLQI), visual analog scales for Ps-related (VAS-Ps) and psoriatic arthritis-related (VAS-PsA) pain, Short Form 36 Health Survey Mental (MCS) and Physical (PCS) Component Summary scores, and Total Activity Impairment (TAI) scores.

Treatment of psoriasis may also entail achieving or maintaining a minimum clinically important difference (MCID) for any of the HRQOL outcomes provided herein, e.g., any one or combination of DLQI, VAS-Ps, VAS-PsA, MCS, PCS and TAI.

Treatment of psoriasis may also entail achieving or maintaining a minimum clinically important difference (MCID) response rate for any of the HRQOL outcomes provided herein, e.g., any one or combination of DLQI, VAS-Ps, VAS-PsA, MCS, PCS and TAI. “Treatment of” or “treating” psoriasis may also mean achieving or maintaining a clinically meaningful reduction in any of the HRQOL outcomes provided herein, e.g., any one or combination of DLQI, VAS-Ps, VAS-PsA, MCS, PCS and TAI. “Treatment of” or “treating” psoriasis may also mean achieving or maintaining a Nail Psoriasis Severity Index (NAPSI) score for a period of time during or following treatment (e.g., for at least 2, 4, 6, 8, 10, 12, 14, 16, 18, 20, 22, 24, 26, 28, 30, 32, 34, 36, 38, 40, 42, 46, 48, 50, 52, 54, 56, 58 or 60 weeks or longer).

Treatment of psoriasis may also entail achieving or maintaining any of the outcomes provided herein in a certain percentage of a population of subjects (e.g., in at least about 10%, 15%, 20%, 25%, 30%, 35%, 40%, 45%, 50%, 55%, 60%, 65%, 70%, 75%, 80%, 85%, 90%, 95%, 99%, or 100% of a population of subjects).

Dosage regimens may be adjusted to provide the optimum desired response (e.g., a therapeutic or prophylactic response). For example, a single dose may be administered, several divided doses may be administered over time or the dose may be proportionally reduced or increased as indicated by the exigencies of the therapeutic situation. The dose may be administered to the subject upon symptoms of skin disease, or before onset of symptoms.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In one embodiment, the dose amount is a fixed dose, e.g., is not dependent on the weight of the subject to which the substance is administered. In another embodiment, the dose amount is not a fixed dose, e.g., is dependent on the weight of the subject to which the substance is administered, or for a topical therapy a dose may be related to the surface area that is treated, e.g. dose/m² of skin.

Exemplary dose amounts, e.g., fixed dose amounts, for use in treating an adult human may include about 0.01 mg, about 0.05 mg, about 0.1 mg, about 0.5 mg, about 1 mg, about 5 mg, about 10 mg, about 50 mg, about 100 mg, about 500 mg, or more.

Exemplary dose amounts, e.g., dose amounts for topical use treating an adult human by the methods of the invention include about 0.01 mg/m² surface area, about 0.05 mg/m² surface area, about 0.1 mg/m² surface area, about 0.5 mg/m² surface area, about 1 mg/m² surface area, about 5 mg/m² surface area, about 10 mg/m² surface area, about 50 mg/m² surface area, about 100 mg/m² surface area, about 500 mg/m² surface area, or more.

Ranges intermediate to the above-recited ranges are also contemplated. For example, ranges having any one of these values as the upper or lower limits are also intended to be part of the invention, e.g., from about 0.01 mg to about 100 mg, from about 1 mg to about 10 mg, etc.

The administration of the composition may comprise a recurring cycle of administration of composition to the subject. The periodicity of administration of the composition may be about once a week, once every other week, about once every three weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks, about once every 21 weeks, about once every 22 weeks, about once every 23 weeks, about once every 24 weeks, about once every 5-10 days, about once every 10-20 days, about once every 10-50 days, about once every 10-100 days, about once every 10-200 days, about once every 25-35 days, about once every 20-50 days, about once every 20-100 days, about once every 20-200 days, about once every 30-50 days, about once every 30-90 days, about once every 30-100 days, about once every 30-200 days, about once every 50-150 days, about once every 50-200 days, about once every 60-180 days, or about once every 80-100 days. Periodicities intermediate to the above-recited times are also contemplated by the invention. Ranges intermediate to the above-recited ranges are also contemplated by the invention. For example, ranges having any one of these values as the upper or lower limits are also intended to be part of the invention, e.g., from about 110 days to about 170 days, from about 160 days to about 220 days, etc.

A duration of the periodicity of administration of a substance may be may be up to about 4 weeks, up to about 8 weeks, up to about 12 weeks, up to about 16 weeks or more, up to about 20 weeks, up to about 24 weeks, up to about 28 week, up to about 32 weeks or more, during which the periodicity of administration is about once every week. For example, a duration of the periodicity may be about 6 weeks during which the periodicity of administration is about once every 4 weeks, e.g., the substance is administered at week zero and at week four.

### EXAMPLES

The abbreviations of Table 1 are used throughout the disclosure.

TABLE 1

| General abbreviations | | Solvent abbreviations | |
|---|---|---|---|
| AAC | Accelerated | ACN | Acetonitrile |
| | Ageing Conditions | DME | 1,2-Dimethoxyethane |
| | (40° C./75% RH) | DMSO | Dimethyl sulfoxide |
| Am | Amorphous | EtOH | Ethanol |
| Amb | Ambient-dried solid sample | FaSSIF | Fasted State |
| API | Active Pharmaceutical | | Simulated |
| | Ingredient | | Intestinal Fluid |
| DSC | Differential Scanning | FeSSIF | Fed State Simulated |
| | Calorimetry | | Intestinal Fluid |
| DVS | Dynamic Vapor Sorption | IPA | Isopropanol/ |
| Evap | Solid sample recovered | | 2-Propanol |
| | by evaporation | MEK | 2-Butanone |
| HR-XRPD | High Resolution X-Ray | MeOH | Methanol |
| | Powder Diffraction | SGF | Simulated Gastric |
| HT-XRPD | High Throughput X-Ray | | Fluid |
| | Powder Diffraction | THF | Tetrahydrofuran |
| | Intensity of diffraction peak | TBME | tert-Butyl methyl |
| IDR | Intrinsic Dissolution rate | | ether |
| ML | Mother liquor | | |
| | (liquid phases) | | |
| MS | Mass Spectroscopy | | |
| Pc | Poorly crystalline | | |
| RH | Relative Humidity | | |
| RT | Room Temperature | | |
| SM | Starting Material | | |
| TGA | Thermogravimetric Analysis | | |
| TGMS | Thermogravimetric | | |
| | Analysis coupled with | | |
| | Mass Spectroscopy | | |
| UPLC | Ultra-Performance Liquid | | |
| | chromatography | | |
| Vac | Vacuum-dried solid sample | | |

### Example 1: Preparation of Novel Salts

The thermal stability of benzamil was evaluated in three solvent systems: acetonitrile:water 1:1, ethanol, and 0.1 M HCl. Solutions of benzamil (˜0.5 mg/mL) were prepared in the selected solvents and split over 3 vials. The solutions were stirred at room temperature, 50° C. and 80° C. for one hour and then analyzed by UPLC MS. The chemical purity of benzamil measured in the solutions incubated at 50° C. and 80° C. for 1 h was compared to that measured initially in the starting solutions. Benzamil appeared to be stable upon incubation in ethanol at 50° C. for 1 h, whereas some chemical degradation was observed in the solution incubated at 80° C. Based on these results, the highest temperature applied in further studies was set to 50° C.

The solubility of benzamil free base was estimated qualitatively in ethanol, water, 1,4-dioxane, 1,2-dimethoxyethane, acetonitrile, tert-butyl methyl ether, heptane, ethylacetate, THF, and methanol. Aliquots of solvent were added to approximately 5 mg of benzamil free base until dissolution occurred. When the benzamil did not dissolve for a concentration of approximately 3 mg/mL, the suspensions were incubated at 50° C. for 30 min to investigate the effect of the temperature on the solubility. Benzamil was insoluble in most of the tested solvents, except for methanol and 1,2-dimethoxyethane, where it was slightly soluble. The suspension in THF appeared lighter upon incubation at 50° C. for 30 min, indicating that the high temperature slightly improved the solubility.

Based on the results of the thermal stability and qualitative solubility test, the salt formation experiments were performed by slurry conversion in methanol (MeOH), 1,2-dimethoxyethane (DME) and THF.

Suspensions of benzamil free base were prepared in the three selected solvents and counterions were added in relations 1:0.5, 1:1 and 1:2 (depending on the pKₐ difference) as aqueous solutions. The mixtures were initially heated at 50° C. for 1 h and afterwards cooled down to 5° C.

The precipitated solids were analyzed by high throughput X-ray powder diffractometry (HT XRPD). Liquid phases (both solutions and mother liquors) were evaporated at ambient conditions and the residual solids were analyzed by HT XRPD. 23 novel crystalline phases were identified (probably as single phases). At least one crystalline form was identified by reaction of benzamil with each tested counterion, except for the gluconic acid. The salt formation with lactic acid in DME and THF produced the same crystalline phase (denoted “LAC1”), whereas in MeOH amorphous material was recovered. In salt formation with acetic, benzoic, tartaric, nitric, phosphoric, p-toluenesulfonic and sulfuric acids different XRPD patterns were often identified in the solids isolated from different solvents, suggesting that the salt could be polymorphic. The reaction of benzamil with benzoic, nitric and p-toluenesulfonic acids also produced mixtures of crystalline phases in several cases.

Solids were being exposed to 40° C./75% relative humidity (RH) for 2 days (Accelerated Aging Conditions, AAC) to test the physical stability of the novel identified phases.

Results are presented in Table 2. Benzamil may be referred to in the Tables as “API”.

TABLE 2

| | | | | Concen- | | | | | | Vacuum- | | |
|---|---|---|---|---|---|---|---|---|---|---|---|---|
| | API | | Solvent | tration | | API:CI | | Precip- | Vacuum- | dried | | |
| Exp | Mass | | Volume | (mg/ | Counterion | ratio | CI | itated | dried | solids | | Evap. |
| ID | (mg) | Solvent | (μL) | mL) | (CI) | 1:x | mmol | solid? | solids | (AAC) | Evap. | (AAC) |
| SSm1 | 30.1 | MeOH | 400 | 75.3 | Sulfuric acid | 1.1 | 0.104 | No | — | — | SUL2 | SUL8 |
| | | | | | | | | | | | deliq. | sticky |
| SSm2 | 29.7 | MeOH | 400 | 74.4 | L-Tartaric acid | 1.1 | 0.102 | Yes | TAR1 pc | TAR1 pc | — | |
| SSm3 | 29.9 | MeOH | 400 | 74.7 | Acetic acid | 1.1 | 0.103 | No | — | — | ACA2 | ACA2 |
| SSm4 | 29.8 | MeOH | 400 | 74.6 | Nitric acid | 1.1 | 0.103 | Yes | NIT4 pc | NIT5 | NIT1 + | NIT5 |
| | | | | | | | | | | | NIT5 | |
| SSm5 | 30.0 | MeOH | 400 | 74.9 | p- | 1.1 | 0.103 | Yes | TOS1 | TOS1 | TOS1 + | TOS1 + |
| | | | | | Toluenesulfonic | | | | | | TOS3 | TOS3 |
| | | | | | acid | | | | | | | |
| SSm6 | 30.7 | MeOH | 400 | 76.7 | Phosphoric | 1.1 | 0.106 | Yes | PHO1 pc | PHO1 + | — | |
| | | | | | acid | | | | | PHO2 pc | | |
| SSm7 | 30.1 | MeOH | 400 | 75.3 | Lactic acid | 1.1 | 0.104 | No | — | — | Am | LAC1 |
| SSm8 | 30.2 | MeOH | 400 | 75.6 | Gluconic acid | 1.1 | 0.104 | No | — | — | Am deliq. | Am deliq. |
| SSm9 | 29.8 | MeOH | 400 | 74.5 | Benzoic acid | 1.1 | 0.103 | No | — | — | BNZ3 | BNZ3 |
| SSm10 | 30.5 | MeOH | 400 | 76.4 | Sulfuric acid | 0.55 | 0.053 | Yes | SUL3 | Am deliq. | Am ly | pc ly |
| SSm11 | 30.6 | MeOH | 400 | 76.4 | L-Tartaric acid | 0.55 | 0.053 | Yes | Am | Am deliq. | — | — |
| SSm13 | 30.2 | MeOH | 400 | 75.5 | Sulfuric acid | 2.1 | 0.199 | No | — | — | SUL6 pc | SUL6 |
| | | | | | | | | | | | sticky | deliq. |
| SSm14 | 29.5 | MeOH | 400 | 73.9 | Nitric acid | 2.1 | 0.194 | Yes | NIT1 + | NIT5 | NIT1 | NIT5 |
| | | | | | | | | | NIT3 | | | |
| SSm15 | 30.4 | MeOH | 400 | 76.0 | p- | 2.1 | 0.199 | Yes | TOS1 + | TOS1 + | TOS2a | TOS2b |
| | | | | | Toluenesulfonic | | | | TOS3 | TOS3 | | deliq. |
| | | | | | acid | | | | | | | |
| SSm16 | 30.3 | MeOH | 400 | 75.9 | None | — | — | Yes | 1 (FB) | 1 (FB) | 3 (FB) | 3 (FB) |
| SSm17 | 30.6 | DME | 500 | 61.1 | Sulfuric acid | 1.1 | 0.105 | Yes | SUL4 pc | SUL7 pc ly | SUL5 ly | SUL8 ly |
| SSm18 | 29.5 | DME | 500 | 58.9 | L-Tartaric acid | 1.1 | 0.101 | Yes | TAR1 pc | TAR1 pc | — | |
| SSm19 | 30.4 | DME | 500 | 60.8 | Acetic acid | 1.1 | 0.105 | Yes | ACA1 | ACA1 | ACA2 | ACA2 |
| SSm20 | 29.6 | DME | 500 | 59.1 | Nitric acid | 1.1 | 0.102 | Yes | 4.0° + | ep + | ep + | NIT1 + |
| | | | | | | | | | NIT6 | NIT5 | NIT1 + | NIT5 |
| | | | | | | | | | | | NIT2 | |
| SSm21 | 29.5 | DME | 500 | 59.0 | p- | 1.1 | 0.102 | Yes | TOS1 | TOS1 | TOS1 + | TOS1 + |
| | | | | | Toluenesulfonic | | | | | | TOS3 | TOS3 |
| | | | | | acid | | | | | | | |
| SSm22 | 30.4 | DME | 500 | 60.7 | Phosphoric | 1.1 | 0.105 | Yes | PHO2 | PHO2 | — | — |
| | | | | | acid | | | | | | | |
| SSm23 | 30.4 | DME | 500 | 60.8 | Lactic acid | 1.1 | 0.105 | Yes | LAC1 | LAC1 | — | — |
| SSm24 | 30.1 | DME | 500 | 60.3 | Gluconic acid | 1.1 | 0.104 | Yes | Am deliq. | Am deliq. | — | — |
| SSm25 | 29.9 | DME | 500 | 59.7 | Benzoic acid | 1.1 | 0.103 | Yes | BNZ1 | BNZ1 | BNZ1 + ep | BNZ1 ly |
| SSm26 | 30.6 | DME | 500 | 61.2 | Sulfuric acid | 0.55 | 0.053 | Yes | SUL1 | SUL1 | — | — |
| SSm27 | 30.5 | DME | 500 | 61.1 | L-Tartaric acid | 0.55 | 0.053 | Yes | TAR2 pc | TAR2 pc | — | — |
| SSm29 | 30.1 | DME | 500 | 60.1 | Sulfuric acid | 2.1 | 0.198 | No | Am deliq | SUL8 + ep deliq. | | |
| SSm30 | 30.5 | DME | 500 | 61.0 | Nitric acid | 2.1 | 0.200 | Yes | NIT1 + | NIT5 | NIT1 | NIT1 + |
| | | | | | | | | | NIT6 | | | NIT5 |
| SSm31 | 30.4 | DME | 500 | 60.8 | p- | 2.1 | 0.200 | Yes | TOS1 + | TOS1 + | TOS2a | TOS3 + |
| | | | | | Toluenesulfonic | | | | TOS3 | TOS3 | | ep sticky |
| | | | | | acid | | | | | | | |
| SSm32 | 30.2 | DME | 500 | 60.4 | None | — | — | Yes | 1 | 1 | 1 | 1 |
| SSm33 | 30.0 | THF | 400 | 74.9 | Sulfuric acid | 1.1 | 0.103 | No | — | — | Am deliq | SUL7 |
| SSm34 | 29.6 | THF | 400 | 73.9 | L-Tartaric acid | 1.1 | 0.102 | Yes | TAR1 pc | TAR1 pc | — | — |
| SSm35 | 30.4 | THF | 400 | 76.0 | Acetic acid | 1.1 | 0.105 | Yes | ACA1 | ACA1 | — | — |
| SSm36 | 30.5 | THF | 400 | 76.3 | Nitric acid | 1.1 | 0.105 | Yes | NIT2 | NIT5 | ep + NIT2 | NIT5 |
| SSm37 | 30.3 | THF | 400 | 75.8 | p- | 1.1 | 0.104 | Yes | TOS4b + | TOS1 + | TOS4a + | TOS4a + |
| | | | | | Toluenesulfonic | | | | TOS3 | TOS4b pc | TOS3 | TOS3 |
| | | | | | acid | | | | | | | |
| SSm38 | 29.6 | THF | 400 | 74.0 | Phosphoric | 1.1 | 0.102 | Yes | PHO3 | PHO1 + | — | — |
| | | | | | acid | | | | | PHO2 | | |
| SSm39 | 30.0 | THF | 400 | 75.1 | Lactic acid | 1.1 | 0.103 | Yes | LAC1 | LAC1 | — | — |
| SSm40 | 30.1 | THF | 400 | 75.3 | Gluconic acid | 1.1 | 0.104 | Yes | Am deliq. | Am deliq. | — | — |
| SSm31 | 30.3 | THE | 400 | 75.7 | Benzoic acid | 1.1 | 0.104 | Yes | BNZ2 | BNZ2 | BNZ1 + | BNZ1 + |
| | | | | | | | | | | | ep ly | ep ly |
| SSm42 | 29.8 | THF | 400 | 74.5 | Sulfuric acid | 0.55 | 0.051 | Yes | SUL1 | SUL1 | — | — |
| SSm43 | 29.7 | THF | 400 | 74.2 | L-Tartaric acid | 0.55 | 0.051 | Yes | TAR1 pc | TAR1 pc | — | — |
| SSm45 | 29.8 | THF | 400 | 74.5 | Sulfuric acid | 2.1 | 0.196 | No | — | — | Am deliq | Am deliq |
| SSm46 | 29.8 | THF | 400 | 74.5 | Nitric acid | 2.1 | 0.196 | Yes | NIT1 + | NIT5 | NIT1 | NIT5 |
| | | | | | | | | | NIT2 | | | |
| SSm47 | 30.3 | THF | 400 | 75.7 | p- | 2.1 | 0.199 | Yes | TOS4a + | TOS4a + | TOS3 | TOS3 |
| | | | | | Toluenesulfonic | | | | TOS3 | TOS3 | | |
| | | | | | acid | | | | | | | |
| SSm48 | 29.6 | THF | 400 | 74.0 | None | — | — | Yes | 2 (FB) | 1 + 2 (FB) | 2 (FB) ly | 2 ly(FB) |
| SSm49 | 30 | Meth- | 400 | 75 | Hydrochloric | 2.1 | 0.1970 | Yes | Di-HCl1 | Di- | Di-HCl3 | N/A |
| | | anol | | | acid | | | | | HCl1 + | | |
| | | | | | | | | | | Mono- | | |
| | | | | | | | | | | HCl(A + B) | | |
| SSm50 | 30 | DME | 500 | 60 | Hydrochloric | 2.1 | 0.1970 | Yes | Di-HCl2 | Mono- | Mono-HCl | N/A |
| | | | | | acid | | | | | HCl(A + B) | A + ep | |
| SSm51 | 30 | THF | 400 | 75 | Hydrochloric | 2.1 | 0.1970 | Yes | Di- | Mono- | Di-HCl3 | N/A |
| | | | | | acid | | | | HCl2 + | HCl(A + B) | | |
| | | | | | | | | | Mono- | | | |
| | | | | | | | | | HCl(A + B) | | | |

The notations “deliq.”, “pc”, “Am”, ly” and “ep” stand for “deliquescent”, “poorly crystalline, “amorphous”, “low yield” and “extra peaks”.
The notations “deliq.”, “pc”, “Am”, indicate that the material was deliquescent, poorly crystalline or amorphous, respectively.
The notations “ly” and “ep” indicates that poor amount of material was recovered and that extra peaks not belonging to any of the known phases were identified.
When a single extra peak was identified in the diffractogram, its 2θ value is reported.

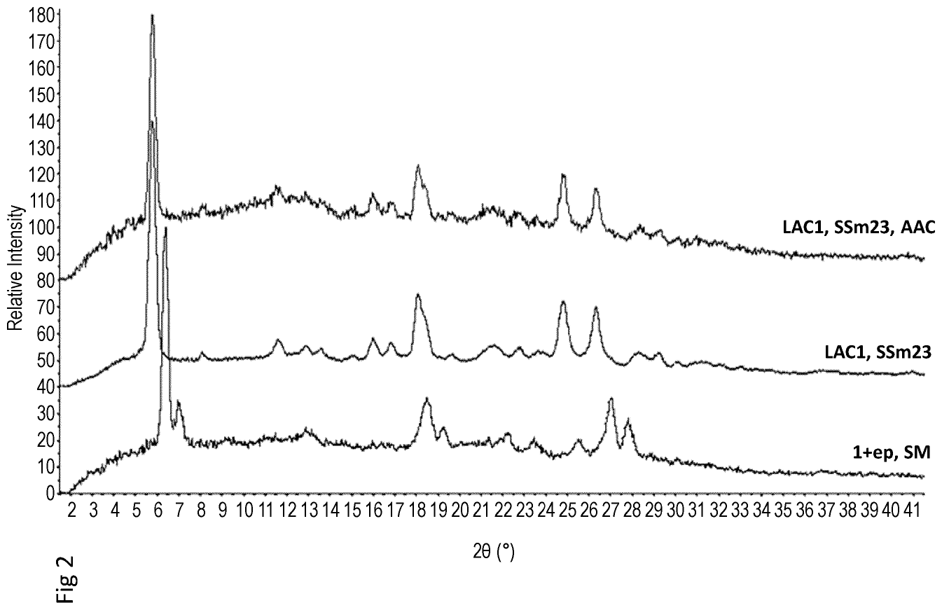

The XRPD patterns for the salts ACA1, ACA2, BNZ1, BNZ2, BNZ3, Di-HC11, Di-HCl2, LAC1, NIT1, NIT2, PHO2, PHO3, TOS1, TOS2a, TOS3, TOS4, TOS5, SUL1, SUL3 showed good crystallinity, while the XRPD patterns for TAR1, TAR2, NIT4, PHO1, SUL2, SUL4, SUL5, SUL6 showed poor crystallinity. FIG. 1 shows an HR-XRPD pattern for the form LAC1 of the lactic acid salt of benzamil. FIG. 2 shows an HR-XRPD pattern for the forms ACA1 and ACA2 of the acetic acid salt of benzamil. FIG. 3 shows an HR-XRPD pattern for the form PHO2 of the phosphoric acid salt of benzamil.

The physically stability test performed at 40° C./75% RH for 2 days indicated that ACA1, ACA2, BNZ1, BNZ2, BNZ3, LAC1, TAR1, TAR2, PHO2, TOS1, TOS3, and SUL1 were physically stable salts, whereas Di-HC11, Di-HCl2, NIT1, NIT2, NIT4, PHO1, PHO3, TOS2a, SUL2, SUL3, SUL4, SUL5, SUL6 were physically unstable salts.

Novel XRPD patterns were distinguished for the salt forms obtained for HCl in methanol and DME. The novel forms were designated “Di-HC11” and “Di-HCl2”. Upon exposure to AAC (40° C./75% RH) for 2 days, both phases partially or fully converted to the mixture of Mono HCl salts A+B (denoted “SM1”). Therefore, both Di-HCl1 and Di-HCl2 were physically unstable.

The aqueous solubility of the salts was estimated qualitatively by the solvent aliquot addition method. Aliquots of water up to 500-600 μL were added to approximately 5 mg of salt. The solubility of the free base was also estimated. Results of the solubility tests are shown in Table 3. LAC1 showed solubility of 26-51 mg/mL; ACA1 and ACA2 showed solubility of 15-19 and 12-16 mg/mL, respectively, and PHO2 showed solubility of 12-16 mg/mL.

The rest of the experiments remained as suspensions (for a concentration of approximately 10 mg/mL). The suspensions were let equilibrating at room temperature for 24 h. Afterwards, solids were separated from the liquid phases. The liquid phases were filtered and analyzed by HPLC to determine the solubility. The pH of solutions/mother liquors was also measured. BNZ1, BNZ2, BNZ3, SUL1, TAR1 pc, TOS1 and NIT5 showed solubility lower than 2 mg/mL. Benzamil free base was practically insoluble in water.

TABLE 3

| | | | API:CI | Physically | | | | | Thermal | API:CI | | |
|---|---|---|---|---|---|---|---|---|---|---|---|---|
| | Solid | | ratio | stable | Poly- | Crystal- | Chemical | Solvation | events | ratio | Water | |
| Exp. ID | form | Salt | 1:x | after AAC | morphism | linity | purity | state | in DCS | (NMR) | solubility | pH |
| Benzamil | 1 + ep | | — | — | Yes | Good | 99.7 | Anhydrous | exo 135; endo | — | <0.1 | 8.1 |
| FB SM2 | | | | | | | | | 216 | | | |
| Benzamil | A + B | Hydro- | 1 | No | Yes | Good | 99.2 | Sesqui- | br. endos 70- | — | 2-3 | 6.6 |
| HCl SM1 | | chloric | | | | | | hydrate | 210; endo 244 | | | |
| | | acid | | | | | | | | | | |
| SSm19.1 | ACA1 | Acetic | 1 | Yes | Yes | Good | 99.5 | Hemi- | brendo 40-70; | 1:1 | 15-19 | 6.4 |
| | | acid | | | | | | hydrate | endo 122 | | | |
| SSm3.1 | ACA2 | Acetic | 1 | Yes | Yes | Good | 99.5 | Mono- | br. endos 40- | 1:1 | 12-16 | 6.6 |
| (Evap.) | | acid | | | | | | hydrate | 130 | | | |
| SSm25.1 | BNZ1 | Benzoic | 1 | Yes | Yes | Good | 98.1 | Anhydrous | endo 198 | 1:1 | 0.4 | 7.5 |
| | | acid | | | | | | | | | | |
| SSm41.1 | BNZ2 | Benzoic | 1 | Yes | Yes | Good | 98.1 | Anhydrous | endo 197 | 1:1 | 0.4 | 7.2 |
| | | acid | | | | | | | | | | |
| SSm9.1 | BNZ3 | Benzoic | 1 | Yes | Yes | Good | 97.2 | Di-hydrate | brendos 50-100; | 1:1.2 | 0.6 | 4.5 |
| (Evap.) | | acid | | | | | | | endo 196 | | | |
| SSm23.1 | LAC1 | L-(+)- | 1 | Yes | No | Good | 99.7 | Anhydrous | endo 190 | 1:1 | 26-51 | 6.1 |
| | | Lactic | | | | | | | | | | |
| | | acid | | | | | | | | | | |
| SSm22.1 | PHO2 | Phosphoric | 1 | Yes | Yes | Good | 100 | Mono- | sm. endos 80- | — | 12-16 | 4.5 |
| | | acid | | | | | | hydrate | 150; endo/exo | | | |
| | | | | | | | | | 170-200; endo | | | |
| | | | | | | | | | 222 | | | |
| SSm26.1 | SUL1 | Sulfuric | 0.55 | Yes | Yes | Poor | 99.8 | Di-hydrate | br. endos 30- | — | 1.5 | 6.6 |
| | | acid | | | | | | | 150 | | | |
| SSm34 | TAR1 | L-Tartaric | 1 | Yes | Unclear | Poor | 99.6 | Sesqui- | br. endos 30- | 1:1 | 1.1 | 3.2 |
| | pc | acid | | | | | | hydrate | 150; exo 188 | | | |
| SSm5.1 | TOS1 | Toluene- | 1 | Yes | Unclear | Good | 99.7 | Anhydrous | sm. exo/endo | 1:1 | 0.2 | 7.4 |
| | | 4-sulfonic | | | | | | | 200-230; endo | | | |
| | | acid | | | | | | | 254 | | | |
| SSm36.1 | NIT5 | Nitric acid | 1 | — | Yes | Good | | Anhydrous | exo 225 | — | 0.6 | 5.7 |
| (AAC) | | | | | | | | | | | | |

Although physically unstable upon AAC (40° C./75% RH) for 2 days, the aqueous solubility was estimated also for the Di-HCl salts in order to have a comparison with the solubility value of 2-3 mg/mL previously estimated for the Mono-HCl salt (Sigma-Aldrich, product number B2417). Suspensions of the two salts were prepared in water with a concentration of 9-10 mg/mL. The mixtures were let equilibrating at RT for 24 h. Afterwards, solids were separated from the liquid phases. The liquid phases were filtered and analyzed by HPLC to determine the solubility. The pH of mother liquors was also measured. The solubility of both salts appeared to be approximately 3 mg/mL, thus being comparable to that of the Mono HCl salt.

The hygroscopic behaviour of the salts was evaluated through Dynamic Vapor Sorption (DVS) measurements with an RH profile 40-95-0-40% at 25° C. and a dm/dt of 0.002. The classification of the hygroscopic behaviour is based on the water vapour uptake at 25° C./80% RH in first adsorption cycle of sorption isotherm. ACA1 and BNZ1 adsorbed 0.1% of water vapour, therefore being non hygroscopic. BNZ2, LAC1, PHO2, TOS1 and NIT5 adsorbed between 0.2 and 0.9% of water vapour, therefore being slightly hygroscopic. The analyzed salts were physically stable upon exposure to variable RH level, since their XRPD pattern was unchanged after the DVS measurement.

Only one polymorph of the lactate salt was found.

In summary, the lactic acid, phosphoric acid, and acetic acid salts of benzamil show improved stability, increased water solubility, demonstrate only slight or no hygroscopicity, and reduced polymorphism.

### Example 2: Preparation of Further Novel Salts

Further salt formation experiments were performed to test additional salts, involving 35 pharmaceutically accepted counterions, in order to test crystallinity, stability and solubility.

### Analytical Methods

The following analytical methods were used in Examples 2 and 3.

### High Throughput X-Ray Powder Diffraction (HT-XRPD)

XRPD patterns were obtained using the Ardena T2 high-throughput XRPD set-up. The plates were mounted on a Bruker General Area Detector Diffraction System (GADDS) equipped with a VÅNTEC-500 gas area detector corrected for intensity and geometric variations. The calibration of the measurement accuracy (peaks position) was performed using NIST SRM1976 standard (Corundum).

Data collection was carried out at room temperature using monochromatic Cu Kα radiation in the 2θ region between 1.5° and 41.5°, which is the most distinctive part of the XRPD pattern. The diffraction pattern of each well was collected in two 2θ ranges (1.5°≤2θ≤21.5° for the first frame, and 19.5°≤2θ≤41.5° for the second) with an exposure time of 90 s for each frame. No background subtraction or curve smoothing was applied to the XRPD patterns.

### Thermal Analysis

TGA/SDTA and TGMS analysis: Mass loss due to solvent or water loss from the crystals was determined by TGA/DSC. Monitoring the sample weight, during heating in a TGA/DSC 3+ STARe system (Mettler-Toledo GmbH, Switzerland), resulted in measure points for a weight vs. temperature curve and a heat flow signal. The TGA/DSC 3+ was calibrated for temperature with samples of indium and aluminum. Samples (circa 2 mg) were weighed into 100 μL aluminum crucibles and sealed. The seals were pin-holed, and the crucibles heated in the TGA from 25 to 300° C. at a heating rate of 10° C. min-1. Dry N₂ gas was used for purging. The gases coming from the TGA samples were analyzed by a mass spectrometer Omnistar GSD 301 T2 (Pfeiffer Vacuum GmbH, Germany), a quadrupole mass spectrometer which analyzes masses in the temperature range of 0-200 amu.

DSC analysis: Thermal events were visualized from DSC thermograms, recorded with a heat flux DSC3+ STARe system (Mettler-Toledo GmbH, Switzerland). The DSC3+ was calibrated for temperature and enthalpy with a small piece of indium (m.p.=156.6° C.; δHf=28.45 J/g) and zinc (m.p.=419.6° C.; δHf=107.5 J/g). Samples (circa 2 mg) were sealed in standard 40 μL aluminum pans, pin-holed and heated in the DSC from 25° C. to 300° C., at a heating rate of 10° C./min. Dry N₂ gas at a flow rate of 50 mL/min was used to purge the DSC equipment during measurement.

### 1H-NMR Spectroscopy

¹H-NMR spectroscopy in DMSO-d₆ was used for compound integrity characterization and to determine the stoichiometry of the salts and cocrystals where appropriate. The spectra were recorded at room temperature (RT) on a 500 MHz instrument (Bruker BioSpin GmbH) using standard pulse sequences. The data was processed with ACD Labs software Spectrus Processor 2016.2.2 (Advanced Chemistry Development Inc. Canada).

### UPLC Analysis

| UPLC System: | | | |
|---|---|---|---|
| UPLC: | Agilent 1290 | | |
| Detector 1: | Diode array UV detector (set at 286 nm) | | |
| Detector 2: | MSD XT single quad in Positive Scan mode | | |
| UPLC Conditions: | | | |
| Auto sampler temp.: | RT | | |
| Column: | Agilent Eclipse Plus C18 HD | | |
| | (50 × 2.1 mm; 1.8 μm) | | |
| Column temp: | 40° C. | | |
| Flow cell: | 10 mm path | | |
| Mobile phase A: | 10 mM Ammonium acetate in Water | | |
| Mobile phase B: | Acetonitrile | | |
| Flow: | 0.6 ml/min | | |
| Gradient: | Time [min]: | Eluent A: | Eluent B: |
| | 0.00 | 95% | 5% |
| | 0.10 | 95% | 5% |
| | 2.50 | 10% | 90% |
| | 2.55 | 10% | 90% |
| | 2.56 | 95% | 5% |
| | 3.50 | 95% | 5% |

| | |
|---|---|
| Run time: | 3.5 min |
| Sample: | |
| Concentration: | appr 1.0 mg/ml |
| Solvent: | MeCN |
| Injection volume: | 1 μl |
| Retention time: | 1.22 min |
| MS: | m/z 320 |

The compound integrity was expressed as a peak-area percentage, calculated from the area of each peak in the chromatogram, except the “injection peak”, and the total peak-area, as follows:

$$peak area ( \% ) = \frac{peak area}{total area of all peaks} \cdot 100 \%$$

The peak area percentage of the compound of interest was employed as an indication of the purity of the component in the sample.

### Experimental Methods

### Materials

Approximately 3 g benzamil lactate salt (batch SBO-84-44) was provided by Ardena, Södertälje, Sweden. All chemicals were obtained from Fisher Scientific or Sigma Aldrich. Chemicals used are of research grade and at least 99% pure.

Free base preparation: Approximately 5 g of benzyl lactate were dissolved in 320 mL of water. The pH of the solution was 5.8. 1 M solution of NaOH in water was added stepwise until the pH was stabilized at 10.5 (in total 14 mL of 1 M NaOH was added) and a white precipitate had formed. The suspension was stirred for 30 min to age the precipitate. The precipitate was filtered over a Buchner funnel and washed twice with 200 mL of water. The obtained solid was dried overnight at 50° C./5 mbar. Yield=3.6 g (92%). The solid was characterized by HT-XRPD, UPLC-MS, TGMS (thermogravimetric analysis coupled with mass spectroscopy) and ¹H-NMR prior to be used as starting material for the salt screen.

### Methods

The salt screen experiments were carried out in methanol (MeOH), 1,2-dimethoxyethane (DME) and tetrahydrofuran (THF). Suspensions of benzamil free base prepared in the three solvents and counterion solutions were added to reach benzamil free base: counter ion molar ratios of: 1:0.55, 1:1.1 and 1:2.2. The counterions of the experiment are listed in Table 4.

TABLE 4
List of the tested pharmaceutically accepted acidic counterions and their
abbreviations (Abbrev.). The GRAS class, pKa values and composition of the
solutions used in the salt screen are also reported for each counterion.

| # | Acids | Abbrev. | Class | pKₐ₁ | pKₐ₂ | pKₐ₃ | Added |
|---|---|---|---|---|---|---|---|
| 1 | Hydrobromic acid | HBR | 3 | <−6 | — | — | 1M (Water) |
| 2 | Naphthalene-1,5-disulfonic acid | NDS | 2 | −3.4 | −2.6 | — | 1M (Water) |
| 3 | Ethane-1,2-disulfonic acid | EDY | 2 | −2.1 | −1.5 | — | 1M (Water) |
| 4 | Methanesulfonic acid | MES | 2 | −1.2 | — | — | 1M (Water) |
| 5 | Naphthalene-2-sulfonic acid | NAS | 2 | 0.2 | — | — | 1M (Water) |
| 6 | Benzenesulfonic acid | BES | 2 | 0.7 | — | — | 1M (Water) |
| 7 | Oxalic acid | OXA | 2 | 1.3 | 4.3 | — | 1M (Water) |
| 8 | L-Aspartic acid | ASP | 1 | 1.9 | 3.7 | 9.6 (b) | 1M (Water) |
| 9 | Maleic acid | MAE | 1 | 1.9 | 6.2 | — | 1M (THF) |
| 10 | Ethanesulfonic | ESY | 2 | 2.1 | — | — | 1M (Water) |
| 11 | Glutamic acid | GLU | 1 | 2.2 | 4.3 | 9.7 (b) | Solid |
| 12 | Pamoic acid | PAM | 2 | 2.5 | 3.1 | — | Solid |
| 13 | Malonic acid | MAO | 2 | 2.8 | 5.7 | — | 1M (Water) |
| 14 | Gentisic acid | GEN | 2 | 2.9 | — | — | 1M (MeOH) |
| 15 | Salicylic acid | SAL | 3 | 3.0 | — | — | 1M (MeOH) |
| 16 | 2-Furoic acid | FUR | — | 3.1 | — | — | Solid |
| 17 | Fumaric acid | FUM | 1 | 3.0 | 4.4 | — | 0.3M (EtOH) |
| 18 | Galactaric acid | GAL | 1 | 3.1 | 3.6 | — | 1M (Water) |
| 19 | Citric acid | CIT | 1 | 3.1 | 4.8 | 6.4 | 1M (Water) |
| 20 | L-Malic acid | MAL | 1 | 3.5 | 5.1 | — | 1M (Water) |
| 21 | Hippuric acid | HIP | 1 | 3.6 | — | — | Solid) |
| 22 | Formic acid | FOR | 3 | 3.8 | — | — | 1M (Water) |
| 23 | L-Ascorbic acid | ASC | 1 | 4.2 | 11.6 | — | 1M (Water) |
| 24 | Succinic acid | SUC | 1 | 4.2 | 5.6 | — | 1M (MeOH) |
| 25 | Glutaric acid | GLT | 1 | 4.3 | 5.3 | — | 1M (Water) |
| 26 | Cinnamic acid | CIN | 3 | 4.4 | — | — | 1M (MeOH) |
| 27 | Adipic acid | ADI | 1 | 4.4 | 5.4 | — | 1M (MeOH) |
| 28 | Valeric acid | VAL | — | 4.8 | — | — | Liquid |
| 29 | Butyric acid | BUT | — | 4.8 | — | — | Liquid |
| 30 | Nicotinic acid | NIC | 2 | 4.9 | 2.1 (b) | — | Solid |
| 31 | Pivalic acid | PIV | — | 4.9 | — | — | Solid |
| 32 | Propionic acid | PRO | 2 | 4.9 | — | — | 1M (Water) |
| 33 | Stearic acid | STE | 1 | 4.9 | — | — | Solid |
| 34 | Orotic acid | ORO | 2 | 5.9 | 9.0 | — | 1M (Water) |
| 35 | Trifluoroacetic acid | TFA | | 0.5 | — | — | 1M (Water) |

The suspensions were heated at 50° C. for 1 h and afterwards cooled down at V° C./h to Y° C. and aged at this temperature for 3 days. The solids were separated from the liquid phases by centrifugation, dried overnight under vacuum at 50° C. and analyzed by HT-XRPD. Solvents from the remaining mother liquors and solutions were evaporated under ambient conditions (evaporative crystallization experiments) and the residual solids were analyzed by HT-XRPD. All solids were exposed to Accelerated Aging Conditions (AAC, 40° C./75% RH) for 2 days and re-analyzed by HT-XRPD.

Counterions, applied benzamil counter ion (API:CI) ratio and isolation conditions (crystallization method and solvent) are reported per each identified salt form, together with an indication of the crystallinity and the physical stability are reported in Table 5.

### Results

TABLE 5
List of benzamil salts identified.

| | Solid | API:Cl | Isolation | | Physical |
|---|---|---|---|---|---|
| Counterion | form | ratio | conditions | Crystallinity | Stability |
| 1,2-Ethanedisulfonic | EDY1 | 1:1.1; | Equilibration in THF | Good | Unstable |
| acid | | 1:0.55 | | | |
| | EDY2 | 1:1.1 | Equilibration in MeOH | Good | Stable |
| | EDY3 | 1:0.55; | Equilibration in DME | Good | Stable |
| | | 1:2.1 | | | |
| | EDY4 | 1:0.55 | Equilibration in MeOH | Good | Unstable |
| | EDY5 | 1:1.1 | After exposure to AAC | Moderate | — |
| | | | from AM | | |
| | EDY6 | 1:0.55 | After exposure to AAC | Good | — |
| | | | from EDY4 | | |
| 1,5- | NDS1 | 1:0.55; | Equilibration in THF, | Good | Stable |
| Naphthalenedisulfonic | | 1:1.1; | DME and MeOH | | |
| acid | | 1:2.1 | | | |
| Gentisic acid | GEN1 | 1:1.1 | Equilibration and | Moderate | Stable |
| | | | evaporation from DME | | |
| | GEN2 | 1:1.1 | Evaporation from THF | Moderate | Stable |
| | GEN3 | 1:1.1 | Equilibration from MeOH | Moderate | Unstable |
| 2-Furoic acid | FUR1 | 1:1.1 | Equilibration in DME | Good | Stable |
| | FUR2 | 1:1.1 | After exposure to AAC | Good | |
| | | | from equilibration and | | |
| | | | evaporation in THF | | |
| | FUR3 | 1:1.1 | Equilibration and | Good | Unstable |
| | | | evaporation from DME | | |
| | FUR4 | 1:1.1 | As a mixture after | Obtained as mixture | Unstable |
| | | | equilibration in THF | | |
| | FUR5 | 1:1.1 | After exposure to AAC | Obtained as mixture | — |
| | | | from AM | | |
| Adipic acid1 | ADI1 | 1:1.1 | Evaporation from THE, | Good | Unstable |
| | | | DME and MeOH | | |
| | ADI3 | 1:1.1 | After exposure to AAC | Good | — |
| | | | from ADI1 | | |
| Benzenesulfonic acid | BES1 | 1:1.1 | Evaporation from THF | Good | Unstable |
| | BES2 | 1:1.1 | Equilibration in MeOH | Good | Unstable |
| | BES3 | 1:1.1 | After exposure to AAC | Good | — |
| | | | from all the systems | | |
| Butyric acid | BUT1 | 1:1.1 | Evaporation from THF | Good | Stable |
| | | | and Equilibration in DME | | |
| | BUT2 | 1:1.1 | Evaporation from MeOH | Poor | Unstable |
| | BUT3 | 1:1.1 | After exposure to AAC | Obtained as a mixture | — |
| | | | from evaporation & | | |
| | | | equilibration in MeOH | | |
| Cinnamic acid | CIN1 | 1:1.1 | Equilibration and | Moderate | Stable |
| | | | evaporation from DME | | |
| | | | and MeOH | | |
| | CIN2 | 1:1.1 | Equilibration in THF | Good | Stable |
| Citric acid | CIT1 | 1:1.1 | Equilibration and | Moderate/Poor | Unstable |
| | | | evaporation from DME | | |
| | | | and MeOH | | |
| | CIT2 | 1:1.1 | After exposure to AAC | Good | — |
| | | | from CIT1 | | |
| Ethanesulfonic Acid | ESY1 | 1:1.1 | Equilibration in MeOH | Good | Stable |
| | ESY2 | 1:1.1 | Evaporation from DME | Obtained as a mixture | Unstable |
| | | | and MeOH | | |
| | ESY3 | 1:1.1 | Evaporation from THF | Poor | Unstable |
| | ESY4 | 1:1.1 | After exposure to AAC | Moderate | — |
| | | | from ESY3 | | |
| Formic acid | FOR1 | 1:1.1 | Equilibration in THF | Good | Unstable |
| | FOR2 | 1:1.1 | Equilibration in MeOH | Moderate/Poor | Unstable |
| | FOR3 | 1:1.1 | After exposure to AAC | Good | — |
| | | | from all the systems | | |
| | FOR4 | 1:1.1 | Evaporation from MeOH | Good | Unstable |
| | FOR5 | 1:1.1 | Evaporation from THF | Good | Unstable |
| Fumaric acid | FUM1 | 1:1.1 | Equilibration in THF and | Good | Unstable |
| | | | DME | | |
| | FUM2 | 1:1.1 | Equilibration in MeOH | Good | Stable |
| | | | and after exposure to | | |
| | | | AAC from FUM1 | | |
| Glutaric acid | GLU1 | 1:1.1 | Equilibration in THF and | Moderate | Stable |
| | | | MeOH. Evaporation from | | |
| | | | THF | | |
| | GLU2 | 1:1.1 | Equilibration in DME | Obtained as mixture | Stable |
| Hippuric acid | HIP1 | 1:1.1 | Equilibration in THF | Good | Unstable |
| | HIP2 | 1:1.1 | After exposure to AAC | Poor | — |
| Hydrobromic acid | HBR1 | 1:1.1; | Equilibration in MeOH | Moderate | Stable |
| | | 1:2.1 | | | |
| | HBR2 | 1:1.1; | Equilibration in THF | Moderate | Unstable |
| | | 1:2.1 | | | |
| | HBR3 | 1:2.1 | Equilibration in DME | Moderate | Unstable |
| | HBR4 | 1:1.1; | Evaporation from MeOH | Moderate | Unstable |
| | | 1:2.1 | and THF | | |
| | HBR5 | 1:1.1; | After exposure to AAC | Moderate | — |
| | | 1:2.1 | | | |
| | HBR6 | 1:1.1; | After exposure to AAC | Good | — |
| | | 1:2.1 | | | |
| | HBR7 | 1:1.1; | After exposure to AAC | Good | — |
| | | 1:2.1 | | | |
| | HBR8 | 1:1.1 | After exposure to AAC | Good | — |
| | HBR9 | 1:2.1 | After exposure to AAC | Obtained as mixture | — |
| | HBR10 | 1:1.1 | Equilibration in DME | Moderate | Unstable |
| L-Glutamic acid | GLT1 | 1:1.1 | Equilibration in MeOH | Moderate | Stable |
| L-Malic acid | MAL1 | 1:1.1 | Equilibration in THF, | Good | Stable |
| | | | DME and MeOH | | |
| | MAL2 | 1:1.1 | After exposure to AAC | Obtained as mixture | — |
| Maleic acid | MAE1 | 1:1.1 | Equilibration in DME, | Good | Unstable |
| | | | MeOH and THF | | |
| | MAE2 | 1:1.1 | After exposure to AAC | Good | Stable |
| | MAE3 | 1:1.1 | After exposure to AAC | Moderate | Stable |
| Malonic acid | MAO1 | 1:1.1 | Equilibration in MeOH | Good | Stable |
| | MAO2 | 1:1.1 | Equilibration in DME | Obtained as mixture | — |
| | MAO3 | 1:1.1 | Equilibration in THF | Obtained as mixture | |
| | MAO4 | 1:1.1 | After exposure to AAC | Good | — |
| | | | from MAO3 | | |
| Methanesulfonic acid | MES1 | 1:1.1; | Equilibration in THE, | Good | Stable |
| | | 1:2.1 | DME and MeOH | | |
| | MES2 | 1:1.1 | Equilibration in DME and | Obtained as mixture | Unstable |
| | | | THF | | |
| | MES3 | 1:1.1 | After exposure to AAC | Moderate | — |
| | | | from MES2 | | |
| Naphthalene- | NSA1 | 1:1.1; | Equilibration in MeOH; | Good | Unstable |
| 2-sulfonic acid | | 1:2.1 | Equilibration and | | |
| | | | evaporation from DME | | |
| | NSA2 | 1:1.1 | Evaporation from THF | Moderate | Stable |
| | NSA3 | 1:2.1 | After exposure to AAC | Obtained as mixture | — |
| | | | from MeOH | | |
| | NSA4 | 1:1.1 | Equilibration and | Poor | Stable |
| | | | evaporation from MeOH | | |
| Nicotinic Acid | NIC1 | 1:1.1 | Equilibration and | Good | Stable |
| | | | evaporation from THF, | | |
| | | | DME and MeOH | | |
| Orotic acid | ORO1 | 1:1.1 | Equilibration and | Good | Stable |
| | | | evaporation from MeOH | | |
| | ORO2 | 1:1.1 | Equilibration and | Good - poorly | Stable |
| | | | evaporation from MeOH | crystalline after | |
| | | | | exposure to AAC | |
| | ORO3 | 1:1.1 | Equilibration in DME | Poor | Poor- |
| | | | | | Stable |
| Oxalic acid | OXA1 | 1:1.1 | Equilibration in THF, | Moderate | Stable |
| | | | DME and MeOH | | |
| Pamoic acid | PAM1 | 1:1.1 | Equilibration and | Obtained as mixture | Stable |
| | | | evaporation from MeOH | | |
| | PAM2 | 1:1.1 | Evaporation from THF | Poor | Stable |
| | PAM3 | 1:1.1 | Equilibration in DME | Poor | Unstable |
| | PAM4 | 1:1.1 | After exposure to AAC | Poor | — |
| | | | from PAM3 | | |
| Pivalic acid | PIV1 | 1:1.1 | Equilibration in THF, | Good | Stable |
| | | | DME and MeOH | | |
| Propionic acid | PRO1 | 1:1.1 | Equilibration in DME | Good | Unstable |
| | PRO2 | 1:1.1 | After exposure to AAC | Moderate | |
| | | | after equilibration in | | |
| | | | MeOH | | |
| | PRO3 | 1:1.1 | Evaporation from THF | Moderate | Unstable |
| | PRO4 | 1:1.1 | Equilibration in THF and | Obtained as mixture | Unstable |
| | | | Evaporation from MeOH | | |
| | PRO5 | 1:1.1 | After exposure to AAC | Moderate | — |
| | | | from PRO3 | | |
| | PRO6 | 1:1.1 | After exposure to AAC | Obtained as mixture | — |
| Salicylic acid | SAL1 | 1:1.1 | Equilibration in THF | Good | Stable |
| | SAL2 | 1:1.1 | After exposure to AAC | Poor | — |
| | | | from AM made in MeOH | | |
| | SAL3 | 1:1.1 | After exposure to AAC | Moderate | Unstable- |
| | | | from AM made in DME | | Deliq |
| Stearic acid | STE1 | 1:1.1 | Equilibration in DME | Obtained as mixture | Stable |
| | STE2 | 1:1.1 | Evaporation from DME | Poor | Stable |
| Succinic Acid | SUC1 | 1:1.1 | Equilibration in THF, | Good | Stable |
| | | | DME and MeOH | | |
| Trifluoroacetic acid | TFA1 | 1:1.1 | Equilibration and | Good | Stable |
| | | | evaporation from THF, | | |
| | | | DME and MeOH | | |
| Valeric acid | VAL1 | 1:1.1 | Evaporation from THE, | Good | Stable |
| | | | DME and MeOH | | |
| | VAL2 | 1:1.1 | After exposure to AAC | Poor | — |
| | | | from evaporation in | | |
| | | | MeOH | | |

¹After further characterization, ADI2 was classified as physical mixture

No salts were found with ethane L-ascorbic, L-aspartic and galactaric acid. However, for the same counterion, different XRPD patterns were often identified in the solids isolated from the different crystallization solvents, suggesting that the salt in question was polymorphic.

In the cases of 1,5-naphthalenedisulfonic acid (NDS1), L-glutamic acid (GLT1), nicotinic acid (NIC), oxalic acid (OXA1), pivalic acid (PIV1), succinic acid (SUC1) and trifluoroacetic acid (TFA1), only one salt polymorph was found from all three crystallization solvents and those forms were all physically stable upon exposure to ACC.

Two crystalline salt forms were distinguished in the solids isolated with cinnamic acid (CIN1-2), citric acid (CIT1-2), fumaric acid (FUM1-2), glutaric acid (GLU1-2), hippuric acid (HIP1-2), L-malic acid (MAL1-2), stearic acid (STE1 and STE3), adipic acid (ADI1 and ADI3) and valeric acid (VAL1-2). Three crystalline salt forms were identified with gentisic acid (GEN1-3), benzenesulfonic acid (BES1-3), butyric acid (BUT1-3), maleic acid (MAE1-3), methane sulfonic acid (MES1-3), orotic acid (ORO1-3) and salicylic acid (SAL1-3). However, upon further analytical characterization it was discovered that ADI2 and STE3 were physical mixtures of the API and CI.

Several forms were identified by salt formation with 1,2-ethanedisulfonic acid (EDY1-6), 2-furoic acid (FUR1-5), ethanesulfonic acid (ESY1-4), formic acid (FOR1-5), hydrobromic acid (HBR1-10), malonic acid (MAO1-4), naphthalene-2-sulfonic acid (NSA1-4), pamoic acid (PAM1-4) and propionic acid (PRO1-6).

The salt forms EDY1, EDY2, EDY3, EDY4, EDY6, NDS1, FUR1, FUR2, FUR3, ADI1, ADI3, BES1, BES2, BES3, BUT1, CIN2, CIT2, ESY1, FOR1, FOR3, FOR4, FOR5, FUM1, FUM2, HIP1, HBR6, HBR7, HBR8, MAL1, MAE1, MAE2, MAO1, MAO4, MES1, NSA1, NIC1, ORO1, ORO2, PIV1, PRO1, SAL1, SUC1, TFA1 and VAL1 showed good crystallinity. The other salt forms showed moderate or poorly crystalline phases or were not isolated as pure forms, only as mixtures, so crystallinity was not evaluated.

Upon exposure to AAC (40° C./75% RH) for 2 days, only the following solid phases were physically stable salts: EDY2, EDY3, NSD1, GEN1, GEN2, FUR1, BUT1, CIN1, CIN2, ESY1, FUM2, GLU1, GLU2, HBR1, GLT1, MAL1, MAE2, MAE3, MAO1, MES1, NSA2, NSA4, NIC1, ORO1, ORO2, OXA1, PAM1, PAM2, PIV1, SAL1, STE1, STE2, SUC1, TFA1 and VAL1.

In conclusion, benzamil showed a tendency to form physically stable salts.

### Solid-State Characterization of Salts

The salts that were physically stable, phase pure forms with good crystallinity were subjected to physicochemical characterization by UPLC, TGA, DSC and ¹H-NMR. The salts obtained after 1 week at 40° C./75% RH were also analyzed by TGMS. Analysis by TGMS determined the amount and nature of solvent that evaporated upon heating the sample.

### Results

The chemical purity of benzamil in the novel identified salts was between 98.7 and 99.9% (area %). More than one crystalline phase was found for some of the salts, not only anhydrous polymorphs but also hydrates, hemihydrates, dihydrates, solvates and combination of water and solvent in the crystal lattice.

TABLE 6
Classification of the salt forms according to the water/solvent content.

| Classification | Solid phase |
|---|---|
| Anhydrous | NDS1; FUR1; BUT1; CIN1 & CIN2; |
| | ESY1; FOR3; FUM1; GLT1; MAO1; NIC1; |
| | OXA1; PIV1; SUC1; TFA1; VAL1 |
| Hemihydrate | GLU1 |
| Monohydrate | HBR1; MAL1; MAO4—with residual |
| | THF; NSA4; ORO1; PRO2 |
| Dihydrate | MES1 |
| Mixed hydrate/non- | EDY3—H₂O and 0.5 equivalents DME |
| stoichiometric solvate | |
| Solvate | GEN1—DME solvate |
| | CIT1—MeOH solvate |
| | FOR1—THF solvate |
| | HIP1—THF solvate |
| | ORO2—THF solvate |
| | SAL1—THF solvate |

The DSC curves usually showed one single event for the anhydrous salts that likely corresponded to the melting and/or starting of the decomposition. For the hydrated/solvated forms, broad endothermic events were recorded before the final melting/starting of decomposition which were assigned to the evaporation of the water and/or solvent from the crystal lattice.

Among the anhydrous salts, the 1,5-napadisylate salt NDS1 showed the highest melting point (280° C.), followed by the esylate salt ESY1 (260° C.), the fumarate salt FUM1 (253° C.), the succinate salt SUC1 (241° C.) and the TFA salt TFA1 (239° C.).

In most of the cases the salt formation was confirmed by ¹H-NMR analysis. Benzamil signals (especially the CH2 group) were found shifted in the spectra of the salts, suggesting that proton transfer had occurred from the acidic counterion to the basic benzamil free base. The stoichiometry of the salt was calculated by proton signal integration.

Most of the salt forms had a 1:1 stoichiometric ratio between benzamil (API) and counter ion, except for MAL1, FUM1 and EDY3 in which the stoichiometric ratio API:CI was 1:0.5. Moreover, for CIT1, HBR1, OXA1 and TFA1 it was not possible to determine the stoichiometry as either the counter ion did not show in the ¹H-NMR spectra or the signals overlaps with the signal of water.

Some salts did not show any shift in their signals (BUT1, CIN1, CIN2, GLU1, PIV1 and VAL1). As can be seen in Table 7, most of those salt forms showed a single event in the DSC trace which most likely corresponded to the melting of the salt, so even though the ¹H-NMR analysis could not confirm salt formation they can be classified as salts. The lack of movement in the NMR signals can be explained by the small difference in their pKa values.

TABLE 7
Classification of the salts according to the water/solvent content

| | | | CI melting | Stoichiometric |
|---|---|---|---|---|
| Salt | DSC results | Classification | point | ratio |
| BUT1 | 186.9 (m/d) | Anhydrous | Liquid | Not shifted—1:1 |
| CIN1 | 217.1 (m/d) | Anhydrous | 133° C. | Not shifted—1:1 |
| CIN2 | 207.1 (m), 208.6 (d) | Anhydrous | 133° C. | Not shifted—1:1 |
| GLU1 | 40-100 (br), | Hemi- | 97.5-98° C. | Not shifted—1:1 |
| | 130.1-134.8-150.0 | hydrate | | |
| | (en-ex-en), 193 (sh) | | | |
| PIV1 | 208.1 (m/d) | Anhydrous | 32-35° C. | Not shifted—1:1 |
| VAL1 | 203.6 (m/d) | Anhydrous | Liquid | Not shifted—1:1 |

To further rank the salts, the aqueous solubility was determined for the anhydrous and hydrated salt forms, as well as for the salts found after the stability study. Given the presence of organic solvent in the solid phase in GEN1, CIT1, FOR1, HIP1, ORO2 and SAL1, no further investigations were carried out on those salts. Additionally, not enough solid of FUR3, ESY1, MAE2 and PAM2 was recovered during the screening so no further studies could be carried out on these salts. Table 8 provides an overview of the physicochemical characterization results collected for the tested salts and their crystalline phase discovered in the screen.

TABLE 8
Summary of solid-state characterization results for the novel physically stable salt
forms. The abbreviations “sm”, “br”, “endo” and “exo” stand for small, broad, endothermic
and exothermic events.

| | | Mass | | | |
|---|---|---|---|---|---|
| Solid | Chemical | loss | | | API:CI |
| form* | Purity (%) | (%) | Solvation state | Thermal events | ratio |
| EDY3 | 99.6 | 4.0 | Monohydrate + DME | 56.1 (br), 103.8 (br), 125-180 (d) | 1:0.5 |
| | | | solvate (0.5 eq) | | |
| NDS1 | 99.7 | 1.1 | Anhydrous | 280.6 (m) | 1:1 |
| GEN1 | 99.4 | 7.9 | DME solvate (0.5 eq) | 153.4 (br) | 1:1 |
| FUR1 | 99.5 | 1.7 | Anhydrous | 217.9 (m), 221.9 (d) | 1:1 |
| BUT1 | 99.9 | 1.1 | Anhydrous | 186.9 (m/d) | Not shifted—1:1 |
| CIN1 | 99.6 | 1.1 | Anhydrous | 217.1 (m/d) | Not shifted—1:1 |
| CIN2 | 99.2 | 0.3 | Anhydrous | 207.1 (m), 208.6 (d) | Not shifted—1:1 |
| CIT1 | 99.7 | 3.2 | MeOH solvate | 90-200 (br) | Overlaps |
| ESY1 | 99.9 | 0.7 | Anhydrous | 259.9 (m) | 1:1 |
| FOR1 | 99.8 | 6.1 | THE solvate (0.3 eq) | 149.2 (en), 156.2 (ex), 218 (m/d) | 1:1 |
| FOR3 | Not | <0.1 | Anhydrous | Not analyzed | — |
| | analyzed | | | | |
| FUM1 | 99.8 | 3.0 | Anhydrous | 194.6 (sm), 252.7 (m/d) | 1:0.5 |
| GLU1 | 99.6 | 0.5 | Hemi-hydrate | 40-100 (br), 130.1-134.8-150.0 | Not shifted—1:1 |
| | | | | (en-ex-en), 193 (sh) | |
| HIP1 | 99.7 | 10.0 | THE solvate (0.7 eq) | 126.1 (br), 134.8 (br) | 1:1 |
| HBR1 | 99.7 | 4.8 | Monohydrate | 80-150 (br), 163.9 (ex), 256.8 (m/d) | Not possible |
| GLT1 | 98.7 | 1.4 | Anhydrous | 204.4 (m/d) | 1:1 |
| MAL1 | 99.4 | 2.4 | Hemihydrate | 91.1 (br), 130.1 (br), | 1:0.5 |
| | | | | 153.8 (br), 178 (d) | |
| MAO1 | 99.8 | 2.2 | Anhydrous | 163.4 (sm), 181.5 (m) | 1:1 |
| MAO4 | Not | 3.8 | Monohydrate/ | Not analyzed | |
| | analyzed | | Residual THF | | |
| MES1 | 99.6 | 9.1 | Dihydrated | 25-1215 (br), 212.2 (m/d) | 1:1 |
| NSA4 | 99.8 | 4.7 | Monohydrate | 99.0 (br), 120.9 (sh), | 1:1 |
| | | | | 173.6 (ex), 201.7 | |
| | | | | (sh), 240.1 (ex) | |
| NIC1 | 99.8 | 2.3 | Anhydrous | 204.8 (m/d) | 1:1 |
| ORO1 | 99.6 | 3.6 | Monohydrate | 25-70 (br), 90-160 (br), | 1:1 |
| | | | | 199.8 (br), 255 (d) | |
| ORO2 | 99.5 | 10.5 | THE solvate (0.7 eq) | 182.3 (br) | 1:1 |
| OXA1 | 99.5 | 2.0 | Anhydrous | 232.2 (m/d) | Overlaps |
| PAM2 | Low yield | | | | |
| PIV1 | 99.9 | 1.7 | Anhydrous/residual | 208.1 (m/d) | Not shifted—1:1 |
| | | | DME | | |
| PRO2 | Not | 4.6 | Monohydrate | Not analyzed | |
| | analyzed | | | | |
| SAL1 | 99.7 | 13.5 | THE solvate (1 eq) | 80-140 (br), 148.9 (ex), 162.8 (sh), | 1:1 |
| | | | | 179.2 (m) | |
| SUC1 | 99.4 | 1.1 | Anhydrous | 241.0 (m/d) | 1:1 |
| TFA1 | 99.9 | <0.1 | Anhydrous | 239.0 (m/d) | Not possible |
| VAL1 | 99.6 | 1.1 | Anhydrous | 203.6 (m/d) | Not shifted—1:1 |

| | |
|---|---|
| EDY2 | Obtained after 1 week at 40° C./75%—Not analyzed |
| EDY6 | Obtained after 1 week at 40° C./75%—Not analyzed |
| GEN2 | Obtained after 1 week at 40° C./75%—Not analyzed |
| FUR3 | Obtained after 1 week at 40° C./75%—Not analyzed |
| ADI3 | Obtained after 1 week at 40° C./75%—Not analyzed |
| BES3 | Obtained after 1 week at 40° C./75%—Not analyzed |
| CIT2 | Obtained after 1 week at 40° C./75%—Not analyzed |
| FUM2 | Obtained after 1 week at 40° C./75%—Not analyzed |
| MAE2 | Obtained after 1 week at 40° C./75%—Not analyzed |
| MAE3 | Obtained after 1 week at 40° C./75%—Not analyzed |
| PAM4 | Obtained after 1 week at 40° C./75%—Not analyzed |

*Full name of counterion is given in Table 4.

### Aqueous Solubility

Aqueous solubility was determined for the anhydrous and hydrated salt forms, as well as for the salts found after the stability study. Aliquots of water were added to the solid salts until dissolution occurred or 1600 μL of water was added. The lactate salt was taken along in the solubility studies as a reference. Full dissolution occurred for MES1, GLT1 and BUT1. All other salt forms did not dissolve completely after addition of 1600 μL of water and remain suspensions. To facilitate full dissolution, these suspensions were heated to 50° C. for 15 min. HBR1, MAO1, FOR3 and PRO2 dissolved under these conditions. The results (in mg/mL free base dissolved for better comparison between salt forms) of the solubility determination performed in water are reported in Table 9.

Upon completion of the solubility determination, the remaining solids were separated from the liquid phases and allowed to air dry. Dried solids were analyzed by HT-XRPD.

TABLE 9
Results of aqueous solubility determination
at RT for a selection of salts.

| | | Dissolved | | |
|---|---|---|---|---|
| Solid | Dissolved | at 50° C., | Concentration | |
| form | at RT? | 15 min | FB [mg/ml] | XRPD |
| HBR1 | No | Yes | ≈3 | No solid |
| NDS1 | No | No | <3 | NDS1 |
| EDY3 | No | No | <3 | EDY3 + |
| | | | | EDY5 |
| EDY2 | No | No | <3 | EDY6 |
| EDY6 | No | No | <3 | EDY6 |
| MES1 | Yes | — | Between | MES1 + |
| | | | 100-50 | MES5^{a} |
| | | | crashed out | |
| NSA4 pc | No | No | <3 | NSA1 + |
| | | | | NSA2 |
| BES3 | No | No | <3 | BES3 |
| OXA1 | No | No | <3 | OXA1 |
| MAE3 | No | No | <3 | MAE3 |
| GLT1 | Yes | — | Between 12-8 | No solid |
| MAO1 | No | Yes | ≈3 | ly—MAO4 |
| MAO4 | No | No | <3 | ly—MAO4 |
| GEN2 | No | No | <3 | ly—GEN2 |
| | | | | pc |
| FUR1 | No | No | <3 | ly—FUR1 |
| | | | | (pc) |
| FUM1 | No | No | <3 | FUM2 |
| FUM2 | No | No | <3 | ly—FUM2 |
| CIT2 | No | No | <3 | CIT2 |
| MAL1 | No | No | <3 | MAL1 |
| SUC1 | No | No | <3 | ly—SUC1 |
| FOR3 | Almost | Yes | ≈3 | No solid |
| GLU1 | Almost | No | <3 | GLU3 |
| CIN1 | No | No | <3 | CIN1 |
| CIN2 | No | No | <3 | CIN3ª |
| ADI3 | No | No | <3 | ADI2 |
| VAL1 | No | No | <3 | VAL1 pc |
| BUT1 | Yes | — | Between 3-5 | No solid |
| NIC1 | Almost | No | <3 | NIC2^{a} |
| PIV1 | No | No | <3 | ly—PIV1 |
| PRO2 | Almost | Yes | ≈3 | No solid |
| STE3 | No | No | <3 | STE2 + |
| | | | | STE3 |
| ORO1 | No | No | <3 | ORO4^{a} |
| TFA1 | No | No | <3 | TFA1 |
| PAM4 | No | No | <3 | PAM1 + |
| | | | | PAM0 |
| LAC1—SM (ref) | Yes | — | ≈20 | — |

| | | |
|---|---|---|
| Mono-HCl (ref) | See Example 1, | 2.4-3.0 |
| | table 2 | |

^{a}new polymorphs found after the water solubility assessment.

Only MES1 showed a better solubility value than the starting material (LAC1). However, precipitation was observed after full dissolution. The aqueous solubility determination was repeated but this time aiming for a concentration of 50 mg/ml. The salt quickly dissolved at RT and after 15 min it crashed out again as a white solid.

### Example 3: Comparative Study of the Lactate and Tri-Fluoroacetic Acid Salts of Benzamil

In this example, the lactate salt and the TFA salt of benzamil were compared with respect to physicochemical parameters, polymorphism, solubility and dissolution rate.

### Materials

Chemicals were obtained from Fisher Scientific or Sigma Aldrich. Chemicals used were of research grade and at least 99% pure. Psomri provided 5 grams of lactate PSM001 (Benzamil lactate, batch SB084-44). Benzamil lactate (in the anhydrous form LAC1), was confirmed by ¹H-NMR. The counter-ion of the lactate salt is visible by ¹H-NMR, and the stoichiometry of the salt was confirmed to be 1:1. A UPLC analysis confirmed the chemical purity to be 99.4% (area %).

### Experimental Methods

Free base preparation: Approximately 2 g of benzamil lactate were dissolved in 128 mL of water. The pH of the solution was 5.8. A 1 M solution of NaOH in water was added stepwise until the pH was stabilized at 10.5 (in total 7 mL of 1M NaOH was added) and a white precipitate had formed. The suspension was stirred for 30 min to age the precipitate. The precipitate was filtered over a Buchner funnel and washed twice with 200 mL of water at RT. The obtained solid was dried overnight at 50° C./5 mbar. Yield was 1.4 g (72%) of the benzamil free base. The solid was analyzed by HT-XRPD and ¹H-NMR and compared against the starting material to ensure the free base formation.

TFA salt preparation: A suspension of the free base (1.2 grams) was prepared in THF (14.75 ml) at RT. The counterion solution (1M TFA in water) was added until an API:CI ratio 1:1.1 was reached. The suspension was heated to 50° C. and held at this temperature for 1 h, subsequently cooled to 5° C. and kept at this temperature for 3 days. Upon completion of the aging time, solid was separated from the liquid phase by centrifugation and analyzed by HR-XRPD, TGA, DSC, UPLC and ¹H-NMR as a vacuum-dried solid (Sample ID: GEN8); confirming the product as the benzamil TFA salt.

Generation of amorphous benzamil lactate salt: Approximately 10 mg of API was dissolved in mixtures of organic solvents and water (listed in Table 10). The solutions were frozen in liquid nitrogen and dried in a Freeze Dryer (Christ Alpha 2-4 LD) overnight. The recovered solids were analyzed by HT-XRPD.

Amorphous solids were obtained from tert-butanol/water (50/50), 1,4-dioxane/water (50/50), tetrahydrofuran/water (50/50), acetonitrile/water (50/50) and 2,2,2-trifluoroethanol/water (50/50). Amorphous solids were analyzed by TGMS to determine the residual solvent/water content. Experimental details and results are reported in Table 10. A larger batch of amorphous material was prepared according to the experimental conditions applied in Exp. ID GEN3 (from 1,4-dioxane/water (50/50), considering that a very high API concentration could be reached in solution and the final preparation contained low residual solvent. The obtained amorphous solid (Exp. ID GEN9) was used for the thermocycling experiments.

TABLE 10
Generation of amorphous phase of benzamil lactate. Solids recovered by
lyophilization were analyzed by HT-XRPD and TGMS. “pc” and “Am”
stand for “poor crystalline” and “Amorphous”.

| | | Concen- | | | Mass |
|---|---|---|---|---|---|
| | | tration | | | loss |
| Exp. ID | Solvent (v/v) | (mg/mL) | Dissolved? | XRPD | (%) |
| GEN2 | Tert-butanol/water | 21.0 | Yes | Am | — |
| | (50/50) | | | | |
| GEN3 | 1,4-Dioxane/water | 51.5 | Yes | Am | 2.8 |
| | (50/50) | | | | |
| GEN4 | Tetrahydrofuran/water | 70.7 | Yes | Am | 4.1 |
| | (50/50) | | | | |
| GEN5 | Acetonitrile/water | 51.5 | Yes | Am | 2.8 |
| | (50/50) | | | | |
| GEN6 | 2,2,2-trifluoroethanol/ | 105.0 | Yes | Am | — |
| | water (50/50) | | | | |
| GEN7 | Ethanol/water (50/50) | 26.5 | Yes | LAC1 pc | — |
| GEN9 | 1,4-Dioxane/water | 50.0 | Yes | Am | 2.3 |
| | (50/50) | | | | |

Generation of amorphous benzamil TFA salt: Approximately 10 mg of TFA salt (from Exp. ID GEN8) was dissolved in mixtures of organic solvents and water (listed in Table 11). The solutions were frozen in liquid nitrogen and dried in a Freeze Dryer (Christ Alpha 2-4 LD) overnight. The recovered solids were analyzed by HT-XRPD. Amorphous solids were obtained from 1,4-dioxane/water (70/30), tetrahydrofuran/water (50/50) and acetonitrile/water (70/30). The amorphous solids were analyzed by TGMS to determine the residual solvent/water content. Experimental details and results are reported in Table 11. A larger batch of amorphous material was prepared according to the experimental conditions applied in Exp. ID GEN11 (from 1,4-dioxane/water (70/30), considering that the highest API concentration was reached in solution and the final preparation contained low residual solvent. The obtained amorphous solid (Exp. ID GEN16) was used for the thermocycling experiments.

TABLE 11
Generation of amorphous phase of benzamil TFA salt. Solids recovered by
lyophilization were analyzed by HT-XRPD and TGMS. “pc” and “Am”
stand for “poor crystalline” and “Amorphous”.

| | | Concen- | | | Mass |
|---|---|---|---|---|---|
| | | tration | | | loss |
| Exp. ID | Solvent (v/v) | (mg/ml) | Dissolved? | XRPD | (%) |
| GEN10 | Tert-butanol/Water | 15.0 | No | — | — |
| | (50/50) | | | | |
| GEN11 | 1,4-Dioxane/Water | 40.0 | Yes | Am | 3.8 |
| | (70/30) | | | | |
| GEN12 | Tetrahydrofuran/ | 25.5 | Yes | Am | 5.2 |
| | Water (50/50) | | | | |
| GEN13 | Acetonitrile/Water | 20.6 | Yes | Am | 3.8 |
| | (70/30) | | | | |
| GEN14 | 2,2,2-trifluoroethanol/ | 20.6 | Yes | Am + ep | — |
| | Water (70/50) | | | | |
| GEN15 | Ethanol/Water (50/50) | 14.6 | No | — | — |
| GEN16 | 1,4-Dioxane/Water | 40.0 | Yes | Am | 2.7 |
| | (70/30) | | | | |

### Polymorphic Landscapes

The polymorphic landscapes of both benzamil lactate and TFA salts were evaluated by thermocycling. Slurries of the amorphous solids (GEN9 and GEN16, respectively) were prepared in 15 solvents at room temperature. The suspensions were subjected to a temperature profile, which comprised of three heating and cooling cycles. At the end of the temperature profile, the solids were isolated by centrifugation and dried at ambient conditions and under vacuum at 50° C. Upon completion of all crystallization experiments, all solids were analyzed by HT-XRPD. Subsequently, all solids were exposed to accelerated aging conditions (AAC, 40° C./75% RH) for 48 h before being analyzed by HT-XRPD again.

### Results

The results are provided in Tables 12 and 13 below. Only one polymorph of the lactate salt was found (LAC1), whereas two forms of the TFA salt was found (TFA1 and TFA2).

TABLE 12
Results of thermocycling experiments - lactate salt

| | API | | | Concen- | Solid | HT-XRPD | | | |
|---|---|---|---|---|---|---|---|---|---|
| | Mass | | Volume | tration | after | | Vac. | | Evap. |
| Exp. ID | (mg) | Solvent | (mL) | (mg/mL) | TCP? | Vac. | (AAC) | Evap. | (AAC) |
| TCP1 | 24.9 | TBME | 500 | 49.8 | Yes | LAC1 | LAC1 | — | — |
| TCP2 | 24.9 | Acetone | 600 | 41.5 | Yes | LAC1 | LAC1 | — | — |
| TCP3 | 24.9 | MeOH | 450 | 55.3 | No | — | — | Oil | Oil |
| TCP4 | 24.9 | THF | 600 | 41.5 | Yes | LAC1 | LAC1 | — | — |
| TCP5 | 24.9 | EtOAc | 600 | 41.5 | Yes | LAC1 | LAC1 | — | — |
| TCP6 | 24.9 | EtOH | 600 | 41.5 | Yes | LAC1 | LAC1 | — | — |
| TCP7 | 24.9 | MEK | 600 | 41.5 | Yes | LAC1 | LAC1 | — | — |
| TCP8 | 24.9 | ACN | 600 | 41.5 | Yes | LAC1 | LAC1 | — | — |
| TCP9 | 24.9 | IPA | 600 | 41.5 | Yes | LAC1 | LAC1 | — | — |
| TCP10 | 24.9 | Water | 500 | 49.8 | Yes | ly | ly | LAC1 | LAC1 |
| TCP11 | 24.9 | 1,4-Dioxane | 600 | 41.5 | Yes | LAC1 | LAC1 | — | — |
| TCP12 | 24.9 | DME | 600 | 41.5 | Yes | LAC1 | LAC1 | — | — |
| TCP13 | 24.9 | Acetone/water | 600 | 41.5 | Yes | LAC1 | LAC1 | — | — |
| | | (95/5% v/v) | | | | | | | |
| TCP14 | 24.9 | EtOH/water | 600 | 41.5 | Yes | LAC1 | LAC1 | LAC1 | LAC1 |
| | | (95/5% v/v) | | | | | | | |
| TCP15 | 24.9 | IPA/water | 600 | 41.5 | Yes | LAC1 | LAC1 | — | — |
| | | (95/5% v/v) | | | | | | | |

TABLE 13
Results of thermocycling experiments - TFA salt

| | API | | | Concen- | Solid | HT-XRPD | | | |
|---|---|---|---|---|---|---|---|---|---|
| | Mass | | Volume | tration | after | | Vac. | | Evap. |
| Exp. ID | (mg) | Solvent | (mL) | (mg/mL) | TCP? | Vac. | (AAC) | Evap. | (AAC) |
| TCP16 | 20.04 | TBME | 600 | 33.4 | Yes | TFA1 | TFA1 | — | — |
| TCP17 | 20.04 | Acetone | 600 | 33.4 | Yes | TFA1 | TFA1 | — | — |
| TCP18 | 20.04 | MeOH | 300 | 66.8 | Yes | TFA1 + TFA2 | TFA1 + TFA2 | — | — |
| TCP19 | 20.04 | THF | 600 | 33.4 | Yes | TFA1 | TFA1 | — | — |
| TCP20 | 20.04 | EtOAc | 600 | 33.4 | Yes | TFA1 | TFA1 | — | — |
| TCP21 | 20.04 | EtOH | 500 | 40.1 | Yes | TFA1 | TFA1 | TFA1 | TFA1 |
| TCP22 | 20.04 | MEK | 600 | 33.4 | Yes | TFA1 | TFA1 | — | — |
| TCP23 | 20.04 | ACN | 500 | 40.1 | Yes | TFA1 | TFA1 | — | — |
| TCP24 | 20.04 | IPA | 500 | 40.1 | Yes | TFA1 | TFA1 | — | — |
| TCP25 | 20.04 | Water | 400 | 50.1 | Yes | TFA2 | TFA2 | — | — |
| TCP26 | 20.04 | 1,4-Dioxane | 400 | 50.1 | Yes | TFA1 | TFA1 | — | — |
| TCP27 | 20.04 | DME | 600 | 33.4 | Yes | TFA1 | TFA1 | — | — |
| TCP28 | 20.04 | Acetone/water | 600 | 33.4 | Yes | TFA1 | TFA1 | TFA1 | TFA1 |
| | | (95/5% v/v) | | | | | | | |
| TCP29 | 20.04 | EtOH/water | 400 | 50.1 | Yes | TFA1 | TFA1 | TFA1 | TFA1 |
| | | (95/5% v/v) | | | | | | | |
| TCP30 | 20.04 | IPA/water | 400 | 50.1 | Yes | TFA1 | TFA1 | — | — |
| | | (95/5% v/v) | | | | | | | |

For the lactate salt, LAC1 was the only polymorph found from all the solvent systems used in the screen. LAC1 was also the crystalline phase delivered as starting material. Based on the results, benzamil lactate salt appears not to be polymorphic. LAC1 was physically stable upon exposure to stress conditions.

In the polymorph screen started with the amorphous TFA salt, next to TFA1 (Example 2, see table 7), TFA2 was recovered. TFA2 and a mixture of TFA1+TFA2 were only found from water and MeOH, respectively. All other solvents resulted in TFA1. Both TFA salts (TFA1 and TFA2) were physically stable upon exposure to AAC (no solid form conversion was observed upon exposure to stress conditions, 2 days at 40° C. and 75% RH). Benzamil TFA salt appeared to be polymorphic.

Solids comprised of the three obtained powder patterns (LAC1, TFA1 and TFA2) were further characterized by UPLC, ¹H-NMR, DSC and TGMS analysis.

### Solid-State Characterization

LAC1 (from Exp. ID TCP2), TFA1 (from Exp. ID TCP16) and TFA2 (from Exp. ID TCP25) were characterized by TGMS, DSC, UPLC and ¹H-NMR.

Salt formation was confirmed by ¹H-NMR analysis, as well as the 1-to-1 stoichiometry in the case of LAC1 (TFA not seen on ¹H-NMR). The thermal analyses were indicative of the anhydrous and non-solvated nature of LAC1 (residual solvent content of 0.5%) and that both TFA1 and TFA2 are non-solvated anhydrous forms. The melting temperature and chemical purity of LAC1, TFA1 and TFA2 are found in Table 14.

TABLE 14
Analytical characterization of salt forms.

| | | | Thermal | | | | Chemical |
|---|---|---|---|---|---|---|---|
| | | | events by | Solvent | | Salt | purity by |
| | Exp. | Physically | DSC | content by | | formation | UPLC |
| Salt | ID | stable? | (Tₚₑₐₖ, ° C.) | TGMS | Nature | by ¹H-NMR | (area %) |
| LAC1 | TCP2 | Yes | 196.4 (m) | 0.5% - | Anhydrous | Shifted - | 100% |
| | | | | residual | | 1:1 | |
| | | | | water | | | |
| TFA1 | TCP16 | Yes | 240.4 (m) | 0.3% - | Anhydrous | Shifted | 100% |
| | | | | residual | | | |
| | | | | water | | | |
| TFA2 | TCP25 | Yes | 243.0 (m) | 0.19% - | Anhydrous | Shifted | 100% |
| | | | | residual | | | |
| | | | | water | | | |

(m) indicates melting event

Additional attempts were made to prepare TFA1 to be able to perform additional characterization. However, all attempts led to TFA2. Therefore, the TFA1 salt obtained in the screen was used for the follow-up studies of solubility and physical and chemical stability, and for the intrinsic dissolution rate studies. TFA2 prepared at large scale (Exp. ID GEN8) was used for the follow-up studies (the anhydrous form with the highest melting temperature).

### Solubility Study

The kinetic and thermodynamic solubilities of the lactate (LAC1) and TFA salts (TFA1 and TFA2) were determined in water upon incubation for 1 h and 18 h at 37° C. under continuous magnetic stirring.

Two sets of suspensions at 30 mg/ml of the LAC1, TFA1 and TFA2 salts were prepared in the selected vehicles. The first set of suspensions were equilibrated at 37° C. for 1 h under continuous stirring while the second set was equilibrated at 37° C. for 18 h under continuous stirring. After 10 min incubation, the pH was measured and upon completion of the equilibration times the pH was measured again. The pH values did not change during the incubation times. After completion of the equilibration times, the liquid phases were separated from the solid phases, filtered and analyzed by UPLC for solubility determination. In water, a solubility around 0.7 mg/mL was determined for both incubation times (1 and 18 h) indicating that the maximum solubility was reached already after 1 h at 37° C. Comparing the solubility results of both TFA salts, it became apparent that the solubility of the TFA1 salt is lower than that determined for the TFA2 salt.

The residual solids were harvested and analyzed by HT-XRPD both as ambient- and as vacuum-dried (5 mbar/25° C.) solids. The solubility determination results are shown in Table 15 below.

TABLE 15
Solubility determination.

| | | | | | Solubility | | XRPD | | | |
|---|---|---|---|---|---|---|---|---|---|---|
| | | pH after | | | (mg/mL) | | 1 h | | 18 h | |
| Exp. ID | Medium | 10 min | 1 h | 18 h | 1 h | 18 h | Amb. | Vac. | Amb. | Vac. |
| QSA75/58 LAC1 | Water | 5.9 | 5.8 | 5.8 | 24.3 | 23.6 | LAC1 | LAC1 | Am | LAC1 |
| QSA84/66 TFA2 | Water | 6.0 | 6.2 | 6.1 | 2.6 | 1.6 | TFA2 | TFA2 | TFA2 | TFA2 |
| QSA103/101 TFA1 | Water | 6.1 | 6.2 | 6.2 | 0.7 | 0.7 | TFA1 | TFA1 | TFA1 | TFA1 |

Amb. = Ambient-dried solid samle.
Vac. = Vacuum-dried solid sample

### Intrinsic Dissolution Rate Study

The intrinsic dissolution rates were determined for LAC1 (batch SB084-44), TFA2 (Exp. ID GEN8) and TFA1 (Exp. ID in TCP17) in water and FaSSIF (Fasted State Simulated Intestinal Fluid).

### Materials and Methods

Rotating disc intrinsic dissolution determinations were performed with a μDiss apparatus (Pion, USA) equipped with 6 independent glass fiber probes each connected to a diode area. Before the start of the experiment the probes were calibrated using the spectrum of a Mercury 362 nm pen-ray lamp. Discs were prepared in a passivated aluminum dye with a mini-IDR press (Heath Scientific, UK).

Six dilutions of benzamil lactate salt were prepared in the dissolution media in the range from 0 to 0.5 mg/mL, and the solutions were stirred with a cross-shaped magnetic stirrer. These dilutions were used for the calibration curve which is used for the calculation of the amount of benzamil in solution. Moreover, the solutions were used to select the wavelength at which the samples were to be measured and to select the proper path length.

About 6-10 mg of the different salts (LAC1, TFA1, TFA2) were compressed in a passivated aluminum dye (standardized surface area of 0.071 cm²) with 40 tons of pressure for 1 minute. The dyes were placed in their Teflon holder magnet stirrer and placed into 20 mL glass vials. Before to the sample measurement, a blank of each media was taken in the respective channel. The experiment started by the gentle addition of the pre-warmed dissolution media. Vials were incubated at 37° C. under continuous stirring at 100 rpm for a maximum of 4 h. UV absorption was measured with the in-line probes at regular intervals. Upon completion of the experiment the pH of the solution was determined. Concentrations of API in solution were calculated with use of the calibration curve and plotted against time. Dissolution rates were calculated over the linear parts of the curves. All dissolution rate experiments were done in triplicate except for TFA1 which were done in duplicate due to the lack of material.

### Results

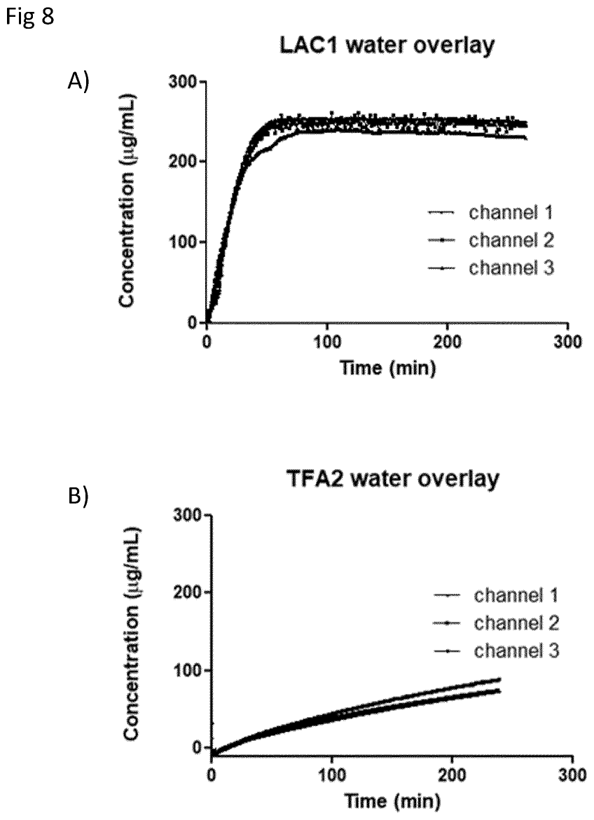

The results are presented in FIG. 8 and Table 16 below. As evident from Table 16 below, the dissolution rate for LAC1 was significantly faster than for TFA2 and TFA1. For LAC1, after about 50 min the whole tablet has been dissolved and the curve becomes flat (FIG. 8A). The IDR of benzmil LAC1 in water was 1.87±0.06 mg/cm²/min calculated over the range from 1 to 36 min.

The dissolution of TFA2 and TFA1 was slower. After about 4 h, the tablet was still not completely dissolved (TFA2: FIG. 8B; TFA1: not shown). The IDR of benzmil TFA2 and TFA1 was 0.13±0.01 and 0.09±0.01 mg/cm²/min (calculated between 10-100 min), respectively.

TABLE 16
Experimental conditions and results for the intrinsic dissolution
rate determinations on benzmil LAC1, TFA2 and TFA1.

| | | Mass tablet | IDR (mg/cm²/min) | |
|---|---|---|---|---|
| Form(s) | Medium | (mg)^{a} | Individual | Average |
| LAC1 | Water | 9.48 | 1.870 | 1.87 ± 0.06 |
| | | 8.07 | 1.811 | |
| | | 12.22 | 1.933 | |
| | FaSSIF | 9.58 | 0.285 | 0.39 ± 0.11 |
| | | 10.34 | 0.510 | |
| | | 10.47 | 0.389 | |
| TFA2 | Water | 23.15 | 0.116 | 0.13 ± 0.1 |
| | | 18.37 | 0.143 | |
| | | 11.24 | 0.130 | |
| | FaSSIF | 13.24 | 0.145 | 0.14 ± 0.004 |
| | | 17.89 | 0.143 | |
| | | 12.45 | 0.138 | |
| TFA1 | Water | 5.19 | 0.082 | 0.09 ± 0.01 |
| | | 6.97 | 0.093 | |
| | FaSSIF | 10.38 | 0.094 | 0.09 ± 0.01 |
| | | 6.08 | 0.076 | |

^{a}After the incubation time, the tablets are still intact

### Conclusion

The Intrinsic Dissolution Rate of LAC1 in water is almost 5-fold faster than the rate in FaSSIF. For TFA1 and TFA2, the Intrinsic Dissolution Rate is between 15 and 20-fold lower than that of LAC1 in water while in FaSSIF the difference is about 4-fold.

### Physio-Chemical Stability Study

Stability studies on benzmil LAC1 (SM, batch SB084-44), TFA2 (from Exp. ID GEN8) and TFA1 (from Exp. ID TCP16) salts were conducted under two stress conditions:
1. Open container at 25° C. and 60% RH
2. Open container at 40° C. and 75% RH

### Materials and Methods

Solids were analyzed by UPLC, TGMS and HT-XRPD after 3 days, 1 week and 3 weeks incubation under stress conditions for LAC1 and TFA2. For TFA1 solids were analyzed after 3 days and 1 week.

### Results

The analytical data is presented in Table 17. No solid form conversion was observed for any of the salts upon incubation in both conditions for 3 weeks, suggesting that both salts were physically stable under the tested conditions.

The chemical purity determined by UPLC for the solids incubated for 3 days, 1 week and 3 weeks at both conditions was comparable to the purity of the solid at the start (t0). This result suggests that no chemical degradation was occurring for any of the salts during the tested conditions.

The thermal analyses (TGMS) showed a small deviation when comparing the t0 and 3 days to the 1 and 3-weeks incubation (from ˜1% to 0.1%). This observation was made for both LAC1 and TFA2. These results could be explained by the presence of traces of residual process solvents in the first two samples (t0 and t3 days) which is released or replaced by water only after 1 week incubation at 25° C./60% RH and 40° C./75% RH.

TABLE 17
HT-XRPD, TGMS and UPLC results of LAC1 (batch SB084-44), TFA2 (from Exp. ID GEN8) andTFA1 (from
Exp. ID TCP16) upon exposure to 25° C./60% RH and 40° C./75% RH for 3 days, 1 week and 3 weeks.

| | | t₀ | | | 3 days | | | 1 week | | | 3 weeks | | |
|---|---|---|---|---|---|---|---|---|---|---|---|---|---|
| Salt | Conditions | XRPD | UPLC | TGMS | XRPD | UPLC | TGMS | XRPD | UPLC | TGMS | XRPD | UPLC | TGMS |
| LAC1 | 25° C. and 60% | LAC1 | 99.4 | 0.4% | LAC1 | 99.4% | 1.2% | LAC1 | 99.4% | <0.1% | LAC1 | 99.4% | <0.1% |
| | RH | | | water | | | water | | | water | | | water |
| LAC1 | 40° C. and 75% | | | | LAC1 | 99.4% | 1.5% | LAC1 | 99.2% | 0.2% | LAC1 | 99.4% | <0.1% |
| | RH | | | | | | water | | | water | | | water |
| TFA2 | 25° C. and 60% | TFA2 | 99.1 | 1.0% | TFA2 | 99.9% | 1.4% | TFA2 | 99.9% | 0.1% | TFA2 | 100% | <0.1% |
| | RH | | | water | | | water | | | water | | | water |
| TFA2 | 40° C. and 75% | | | | TFA2 | 99.9% | 1.2% | TFA2 | 100% | 0.1% | TFA2 | 100% | <0.1% |
| | RH | | | | | | water | | | water | | | water |
| TFA2 | 25° C. and 60% | TFA1 | 100% | 0.3% | TFA1 | 100% | <0.1 | TFA1 | 100% | <0.1 | — | — | — |
| | RH | | | water | | | water | | | water | | | |
| TFA2 | 40° C. and 75% | | | | TFA1 | 100% | <0.1 | TFA1 | 100% | <0.1 | — | — | — |
| | RH | | | | | | water | | | water | | | |

### Conclusion

It was concluded that salts LAC 1, TFA1 and TFA2 are physically and chemically stable upon incubation for 3 weeks at 25° C./60% RH and 40° C./75% RH.

Overall Conclusion from Example 3

Overall, it can be concluded that the lactate salt of benzamil has a significantly higher solubility than the TFA salts.

What is claimed is:

1. A salt of a free base compound represented by formula (I) 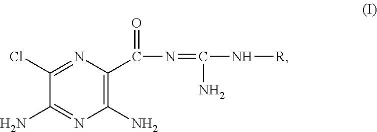
wherein R is selected from 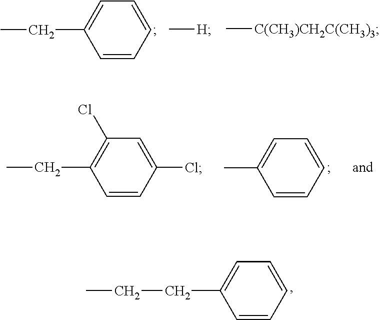
wherein the salt is selected from lactic acid salt, acetic acid salt, or phosphoric acid salt.

2. The salt according to claim 1, wherein the salt is the lactic acid salt.

3. The salt according to claim 1, wherein the free base compound is benzamil.

4. The salt according to claim 1, wherein the salt is in only one crystal form.

5. A pharmaceutical or cosmetic composition comprising:
a salt of a free base compound represented by formula (I) 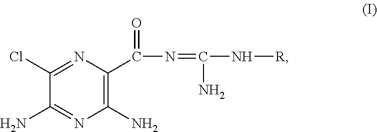
wherein R is selected from 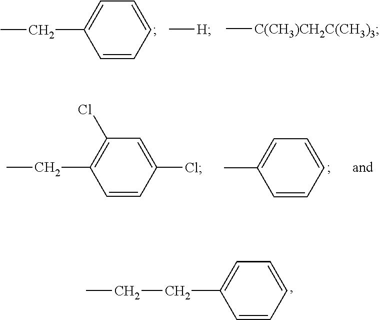
wherein the salt is selected from lactic acid salt, acetic acid salt, or phosphoric acid salt.

6. The composition according to claim 5, wherein the composition is adapted for topical administration.

7. The salt according to claim 1, wherein the salt is for use in medicine.

8. The salt according to claim 7, wherein the use in medicine is a use in a method of treatment of psoriasis.

9. The salt according to claim 8, wherein said psoriasis is chronic psoriasis or plaque psoriasis.

10. The salt according to claim 7, wherein the salt is administered topically or systemically.

11. Use of the salt according to claim 1 in the manufacture of a pharmaceutical composition for use in a method of treatment of psoriasis.

12. A method for treatment of psoriasis comprising administering to an individual in need thereof an effective amount of a salt according to claim 1.

13. The method for treatment of psoriasis according to claim 12, wherein the salt is administered topically.

14. The method for treatment of psoriasis according to claim 12, wherein the psoriasis is chronic psoriasis or plaque psoriasis.

15. The composition according to claim 5, wherein the salt is for use in medicine.

16. The composition according to claim 15, wherein the use in medicine is a use in a method of treatment of psoriasis.

17. The composition according to claim 16, wherein said psoriasis is chronic psoriasis or plaque psoriasis.

18. The composition according to claim 15, wherein the salt is administered topically or systemically.

19. Use of the salt according to claim 2 in the manufacture of a pharmaceutical composition for use in a method of treatment of psoriasis.

20. Use of the salt according to claim 3 in the manufacture of a pharmaceutical composition for use in a method of treatment of psoriasis.
